(19) United States
(12) Patent Application Publication
(10) Pub. No.: US 2026/0232805 A1
(43) Pub. Date: Aug. 13, 2026

(54) METHODS FOR IMAGING USING SPCCT

(71) Applicants: NH THERAGUIX, Meylan (FR); UNIVERSITE CLAUDE BERNARD LYON 1, Villeurbanne (FR); HOSPICES CIVILS DE LYON, Lyon (FR); INSTITUT NATIONAL DE LA SANTÉ ET DE LA RECHERCHE MÉDICALE, Paris (FR); INSTITUT NATIONAL DES SCIENCES APPLIQUEES DE LYON, Villeurbanne (FR); UNIVERSITE JEAN MONNET SAINT-ETIENNE, Saint Etienne (FR); CENTRE NATIONAL DE LA RECHERCHE SCIENTIFIQUE, Paris (FR)

(72) Inventors: François LUX, Villeurbanne (FR); Olivier TILLEMENT, Fontaines Saint-Martin (FR); Salim SI-MOHAMED, Lyon (FR); Philippe DOUEK, Lyon (FR); Loïc BOUSSEL, Lyon (FR); Géraldine LE DUC, Crolles (FR)

(21) Appl. No.: 19/122,480

(22) PCT Filed: Oct. 18, 2023
(86) PCT No.: PCT/EP2023/079045

§ 371 Date: Apr. 18, 2025

(30) Foreign Application Priority Data
Oct. 18, 2022 (EP) 22306578.0

(51) Int. Cl. A61K 41/00; A61K 49/00; A61K 49/08; A61K 49/18

(52) U.S. Cl. CPC A61K 41/0038; A61K 49/0002; A61K 49/085; A61K 49/1824

(57) ABSTRACT

The present invention relates to a method for imaging an anatomical structure in a subject in need thereof, comprising the following steps: a) providing an injectable pharmaceutical composition comprising, as a contrast agent, at least one nanoparticle having a mean hydrodynamic diameter below 10 nm and comprising: a biocompatible matrix, such as polyorganosiloxane, at least one chelating agent covalently bonded to said biocompatible matrix, at least one element having a Z of at least 40, chelated to at least a part of the chelating agents, b) injecting an effective amount of said pharmaceutical to said subject, and, c) acquiring an imaging scan of an anatomical structure of said subject in need thereof, by Spectral Photon Counting Computed Tomography (SPCCT) scanning.

### TECHNICAL FIELD

This disclosure pertains to the field of imaging using a Spectral Photon Counting Computed Tomography (SPCCT) device.

### BACKGROUND

Spectral Photon-Counting Computed Tomography (SPCCT) is emerging as a novel and promising imaging modality in the field of diagnostic radiology. Its energy resolving sensors, known as photon-counting detectors (PCDs), enable the analysis of each photon by dividing them into multiple energy bins. It is therefore possible to perform not only a two-material decomposition (e.g water and iodine) but also K-edge imaging to generate an additional material specific map. K-edge imaging is essential for distinguishing between two contrast agents. This characterization enables to obtain specific information on elements that have a K-edge in the energy range of CT imaging (around 40 to 140 KeV). Taking advantage of the K-edge imaging approach, some animal studies have recently reported that SPCCT can differentiate between two contrast agents in vivo, e.g. by combining them simultaneously within the vascular and peritoneal compartments or by injecting them intravenously. Another significant benefit of spectral imaging is the possibility to measure the absolute concentration of contrast agents.

There is still a need in developing novel contrast agents to optimize and take full benefit of the SPCCT technology. It has surprisingly been found that certain nanoparticles can be used as contrast agent for SPCCT imaging, allowing to increase the acquisition time and obtain images with very high definition and accuracy, opening opportunities for novel imaging applications in particular for diagnostic and theragnostic applications.

### SUMMARY

Accordingly, an embodiment E1 of the present disclosure relates to a method for imaging an anatomical structure in a subject in need thereof, comprising the following steps:
a) providing an injectable pharmaceutical composition comprising, as a contrast agent, at least one nanoparticle having a mean hydrodynamic diameter between 2 nm and 8 nm and comprising:
   a biocompatible matrix, such as polyorganosiloxane,
   at least one chelating agent covalently bonded to said biocompatible matrix,
   at least one element having a Z of at least 40, chelated to at least a part of the chelating agents,
b) injecting an effective amount of said pharmaceutical to said subject, and,
c) acquiring an imaging scan of an anatomical structure of said subject in need thereof, by Spectral Photon Counting Computed Tomography (SPCCT) scanning.

An embodiment E2 of the present disclosure relates to the method according to embodiment E1, wherein said nanoparticles, each comprises:
polyorganosiloxane with a silicon weight ratio of at least 8% of the total weight of the nanoparticle, preferably between 8% and 50%,
chelating agent covalently bound to said polyorganosiloxane, in a proportion comprising between 4 and 200, preferably between 4 and 80 per nanoparticle, and,
at least one element having a Z of at least 40 chelated to at least a part of the chelating agents.

An embodiment E3 of the present disclosure relates to the method according to any one of embodiments E1 or E2, wherein said chelating agent is one or more of DOTA, DTPA, EDTA, EGTA, BAPTA, NOTA, DOTAGA, DFO, DOTAM and DTPABA.

An embodiment E4 of the present disclosure relates to the method according to any one of embodiments E1 to E3, wherein at least one element having a Z of at least 40 is chelated to at least 65% of the chelating agents, preferably at least 75% and more preferably at least 80%.

An embodiment E5 of the present disclosure relates to the method according to any one of embodiments E1 to E4, wherein the element having a Z of at least 40 is selected in the group consisting of lanthanum, cerium, praseodymium, neodymium, promethium, samarium, europium, gadolinium, terbium, dysprosium, holmium, erbium, thulium, ytterbium, lutetium, hafnium, tantalum, tungsten, rhenium, osmium, iridium, platinum, gold and bismuth, preferably in the group consisting of gadolinium and bismuth.

An embodiment E6 of the present disclosure relates to the method according to any one of embodiments E1 to E5, wherein said nanoparticle is gadolinium and bismuth-chelated polyorganosiloxane nanoparticle of the following formula:

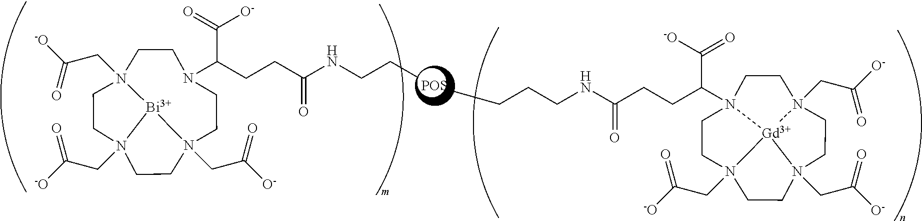

wherein POS is a matrix of polyorganosiloxane, and,
n is comprised between 0 and 100,
m is comprised between 0 and 100,
provided that n+m is comprised between 4 and 200, preferably 4 and 80, and
wherein said nanoparticle has a mean hydrodynamic diameter comprised between 2 and 8 nm, preferably 2 and 6 nm.

An embodiment E7 of the present disclosure relates to the method according to any one of embodiments E1 to E6, wherein said nanoparticle is gadolinium-chelated polyorganosiloxane nanoparticles of the following formula:

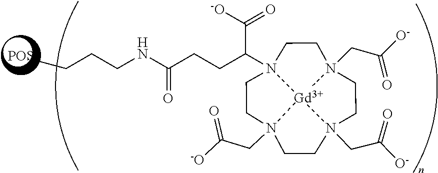

wherein POS is a matrix of polyorganosiloxane, and,
n is comprised between 4 and 200, preferably 4 and 80, and
wherein said nanoparticle has a mean hydrodynamic diameter comprised between 2 and 8 nm, preferably 2 and 6 nm.

An embodiment E8 of the present disclosure relates to the method according to any one of embodiments E1 to E7, wherein said nanoparticle is a lyophilized powder contained in a pre-filled vial to be reconstituted in an aqueous solution for intravenous injection.

An embodiment E9 of the present disclosure relates to the method according to any one of embodiments E1 to E7, wherein said nanoparticle is comprised in an injectable composition at a concentration between 50 and 500 mg/mL, preferably between 80 and 400 mg/mL and more preferably between 100 and 350 mg/mL.

An embodiment E10 of the present disclosure relates to the method according to any one of embodiments E1 to E7 and E9, wherein said nanoparticle is comprised in an injectable composition with a concentration of at least one element having a Z of at least 40 chelated to at least a part of the chelating agents of between 2.5 and 150 mg/mL, preferably between 4 and 120 mg/mL and more preferably between 5 and 105 mg/mL.

An embodiment E11 of the present disclosure relates to the method of any of embodiments E1 to E10, for arterial or veinous angiographic imaging of a subject in need thereof, preferably wherein an efficient amount of said nanoparticle is administered via intra-arterial or intravenous route.

An embodiment E12 of the present disclosure relates to the method of any of embodiments E1 to E10, for renal or urinary system imaging of a subject in need thereof, preferably wherein an efficient amount of said nanoparticle is administered via intra-arterial or intravenous route.

An embodiment E13 of the present disclosure relates to the method of any of embodiments E1 to E10, for cardio-vascular imaging of a subject in need thereof.

An embodiment E14 of the present disclosure relates to the method of embodiment E13, wherein said subject is in need of cardiac catheterism, wherein said nanoparticle is administered via coronarian intraarterial route.

An embodiment E15 of the present disclosure relates to the method of any of embodiments E1 to E10, for hepatic imaging, in particular after intravenous injection.

An embodiment E16 of the present disclosure relates to the method of any of embodiments E1 to E15, wherein at step d) said image is acquired between 5 minutes and 10 minutes.

An embodiment E17 of the present disclosure relates to a composition of nanoparticles having a mean hydrodynamic diameter between 1 nm and 10 nm, comprising:
a biocompatible matrix, such as polyorganosiloxane,
at least one chelating agent covalently bonded to said biocompatible matrix,
at least one element having a Z of at least 40 chelated to at least a part of the chelating agents,
   for its use in a method for diagnosing a condition in a subject in need thereof, said method comprising the step of
a) injecting an effective amount of said nanoparticles to said subject, as contrast agent for imaging,
b) acquiring an image scan of an anatomical structure of the subject in need thereof via SPCCT scanning;
c) analyzing the image scan, thereby diagnosing said condition.

An embodiment E18 of the present disclosure relates to the composition for its use according to embodiment E17, wherein said nanoparticles comprise:
polyorganosiloxane with a silicon weight ratio of at least 8% of the total weight of the nanoparticle, preferably between 8% and 50%,
chelating agents covalently bound to said polyorganosiloxane, in a proportion comprising between 4 and 200, preferably between 4 and 80 per nanoparticle, and,
at least one element having a Z of at least 40 chelated to at least a part of the chelating agents.

An embodiment E19 of the present disclosure relates to the composition for its use according to any one of embodiments E17 or E18, wherein said chelating agent is one or more of DOTA, DTPA, EDTA, EGTA, BAPTA, NOTA, DOTAGA, DFO, DOTAM and DTPABA.

An embodiment E20 of the present disclosure relates to the composition for its use according to any one of embodiments E17 to E19, wherein the element having a Z of at least 40 is selected in the group consisting of lanthanum, cerium, praseodymium, neodymium, promethium, samarium, europium, gadolinium, terbium, dysprosium, holmium, erbium, thulium, ytterbium, lutetium, hafnium, tantalum, tungsten, rhenium, osmium, iridium, platinum, gold and bismuth, preferably in the group consisting of gadolinium and bismuth.

An embodiment E21 of the present disclosure relates to the composition for its use according to any one of embodiments E17 to E20, wherein said nanoparticles are gadolinium and/or bismuth-chelated polyorganosiloxane nanoparticles of the following formula:

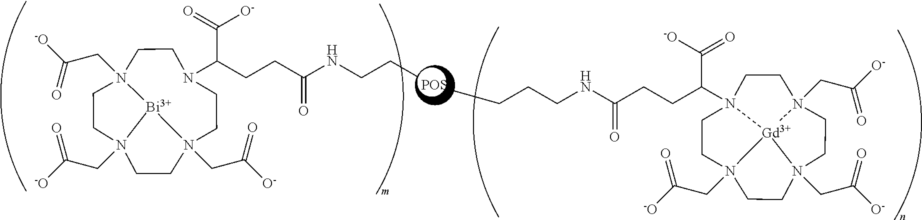

wherein POS is a matrix of polyorganosiloxane, and,
n is comprised between 0 and 100,
m is comprised between 0 and 100,
provided that n+m is comprised between 4 and 200, preferably 4 and 80, and
wherein said nanoparticle has a mean hydrodynamic diameter comprised between 2 and 8 nm, preferably 2 and 6 nm.

An embodiment E22 of the present disclosure relates to the composition for its use according to any one of embodiments E17 to E21, wherein said nanoparticles are gadolinium-chelated polyorganosiloxane nanoparticles of the following formula:

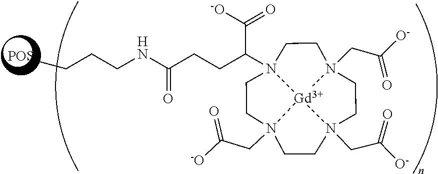

wherein POS is a matrix of polyorganosiloxane, and,
n is comprised between 4 and 200, preferably 4 and 80, and
wherein said nanoparticles have a mean hydrodynamic diameter comprised between 2 and 8 nm, preferably 2 and 6 nm.

An embodiment E23 of the present disclosure relates to the composition for its use according to any one of embodiments E17 to E22, wherein said composition is a lyophilized powder contained in a pre-filled vial to be reconstituted in an aqueous composition for intravenous injection.

An embodiment E24 of the present disclosure relates to the composition for its use according to any one of embodiments E17 to E22, which is an injectable composition of nanoparticles at a concentration between 50 and 500 mg/mL, preferably between 80 and 400 mg/mL and more preferably between 100 and 350 mg/mL.

An embodiment E25 of the present disclosure relates to the composition for its use according to any one of embodiments E17 to E24, wherein the condition is an arterial or venous abnormality, preferably said method comprising an intravenous injection of an aqueous composition of said nanoparticles at a debit of 2 to 3 mL/s, and wherein said composition of nanoparticles comprises between 2.5 and 150 mg/mL of gadolinium atoms, preferably between 4 and 120 mg/mL and more preferably between 5 and 105 mg/mL.

An embodiment E26 of the present disclosure relates to the composition for its use according to any one of embodiments E17 to E24, wherein said condition is a perfusion abnormality selected from vascular abnormalities of the aortic, branches, cardiac, coronarian, pulmonary, cerebroencephalic, digestive, renal and vascular systems.

An embodiment E27 of the present disclosure relates to the composition for its use according to any one of embodiments E17 to E24, wherein said condition is selected from the group consisting of cardiac ischemia, myocardial infarction, and stroke.

An embodiment E28 of the present disclosure relates to the composition for its use according to any one of embodiments E17 to E24, for diagnosing the zone at risk and the size of an infarction before and after revascularization, optionally in combination with iodine contrast agent.

An embodiment E29 of the present disclosure relates to the composition for its use according to any one of embodiments E17 to E24, for diagnosing acute pulmonary embolism, in particular by morphological and functional imaging.

An embodiment E30 of the present disclosure relates to the composition for its use according to any one of embodiments E17 to E24, for diagnosing primary or secondary pulmonary tumors, viral pneumopathies, interstitial fibrosis, or chronic obstructive pulmonary disorders, cystic fibrosis, and bronchiolitis, wherein said nanoparticle is administered via the airways.

An embodiment E31 of the present disclosure relates to the composition for its use according to any one of embodiments E17 to E24, for diagnosing primary or secondary bladder tumor, primary or secondary ureteral tumor primary or secondary renal tumor, lithiasis or renal insufficiency, wherein said nanoparticle is administrated via urinal route.

An embodiment E32 of the present disclosure relates to the composition for its use according to any one of embodiments E17 to E24, wherein said condition is selected from the group consisting of vascular calcification, coronary stenosis or in-stent restenosis.

An embodiment E33 of the present disclosure relates to the composition for its use according to any one of embodiments E17 to E32, wherein an efficient amount of said nanoparticle is administered via intra-arterial route, intravenous, intratumor and intraperitoneal, in particular intraperitoneal.

An embodiment E34 of the present disclosure relates to the composition for its use according to any one of embodiments E17 to E33, wherein said subject is selected among subjects who cannot be treated with iodine as contrast agent.

An embodiment E35 of the present disclosure relates to a composition of nanoparticles having a mean hydrodynamic diameter between 1 nm and 10 nm and comprising:
a biocompatible matrix, such as polyorganosiloxane,
at least one chelating agent covalently bonded to said biocompatible matrix,
at least one element having a Z of at least 40 chelated to at least a part of the chelating agents,
   for its use in a method for diagnosing and treating a tumor in a subject in need thereof, wherein said method comprises at least a first step of SPCCT imaging with said nanoparticle as an imaging contrast agent, a second step of treating said tumor with said nanoparticle as a radiosensitizing agent in combination with radiation therapy.

An embodiment E36 of the present disclosure relates to the composition for its use according to embodiment 35, wherein said tumor is a solid tumor, preferably selected from the group consisting of glioblastoma, brain metastases, meningioma, or primary tumor of uterine cervix, rectum, lung, head and neck, prostate, colorectal, liver, and pancreas cancers.

An embodiment E37 of the present disclosure relates to the composition for its use according to embodiment 35, wherein said tumor is a brain metastases, typically a brain metastases from melanoma, lung, breast, kidney primary cancers.

An embodiment E38 of the present disclosure relates to the composition for its use according to any of embodiments E35 or E37, wherein said nanoparticles is administered via intratumoral route.

An embodiment E39 of the present disclosure relates to a composition of nanoparticles having a mean hydrodynamic diameter between 1 nm and 10 nm and comprising:
a biocompatible matrix, such as polyorganosiloxane,
at least one chelating agent covalently bonded to said biocompatible matrix,
at least one metallic element having a Z of at least 40 chelated to at least a part of the chelating agents,
   for its use in a method for diagnosing and treating cardiac disorders in a subject in need thereof, said method comprising the step of
a) injecting an effective amount of said nanoparticle to said subject, as contrast agent for myocardial imaging,
b) acquiring an image of the myocardium of the subject in need thereof;
c) analyzing the image, and identifying the pathological myocardium, and,
d) irradiating the pathological myocardium by radiotherapy, preferably stereotaxic radiotherapy, in the presence of an effective amount of said nanoparticle as radiosensitizing agent,
e) optionally, acquiring an image scan of the myocardium of the subject in need thereof after step d) in view of evaluating the efficiency and tolerability of the treatment.

An embodiment E40 of the present disclosure relates to the composition for its use according to embodiment E39, wherein at step b) the image is acquired via SPCCT scanning, magnetic resonance imaging (MRI) or SPCCT scanning.

An embodiment E41 of the present disclosure relates to the composition for its use according to any one of embodiments E39 or E40, wherein said nanoparticles, each comprises:
polyorganosiloxane with a silicon weight ratio of at least 8% of the total weight of the nanoparticle, preferably between 8% and 50%,
chelating agents covalently bound to said polyorganosiloxane, in a proportion comprising between 4 and 200, preferably between 4 and 80 per nanoparticle, and,
at least one element having a Z of at least 40 chelated to at least a part of the chelating agents.

An embodiment E42 of the present disclosure relates to the composition for its use according to any one of embodiments E39 to E41, wherein said chelating agent is one or more of DOTA, DTPA, EDTA, EGTA, BAPTA, NOTA, DOTAGA, DFO, DOTAM and DTPABA.

An embodiment E43 of the present disclosure relates to the composition for its use according to any one of embodiments E39 to E42, wherein the element having a Z of at least 40 is selected in the group consisting of lanthanum, cerium, praseodymium, neodymium, promethium, samarium, europium, gadolinium, terbium, dysprosium, holmium, erbium, thulium, ytterbium, lutetium, hafnium, tantalum, tungsten, rhenium, osmium, iridium, platinum, gold and bismuth, preferably in the group consisting of gadolinium and bismuth.

An embodiment E44 of the present disclosure relates to the composition for its use according to any one of embodiments E39 to E43, wherein said nanoparticles are gadolinium and/or bismuth-chelated polyorganosiloxane nanoparticle of the following formula:

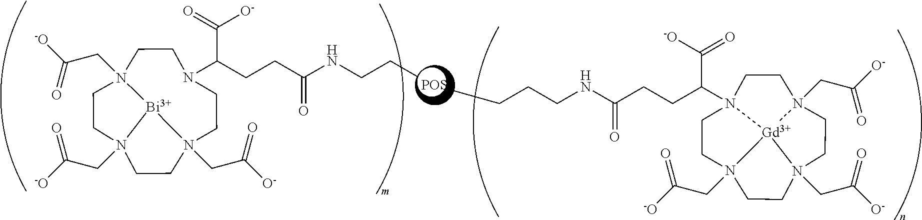

wherein POS is a matrix of polyorganosiloxane, and,
n is comprised between 0 and 100,
m is comprised between 0 and 100,
provided that n+m is comprised between 4 and 200, preferably 4 and 80, and
wherein said nanoparticle has a mean hydrodynamic diameter comprised between 2 and 8 nm, preferably 2 and 6 nm.

An embodiment E45 of the present disclosure relates to the composition for its use according to any one of embodiments E39 to E44, wherein said nanoparticles are gadolinium-chelated polyorganosiloxane nanoparticles of the following formula:
wherein said nanoparticle is gadolinium-chelated polyorganosiloxane nanoparticles of the following formula:

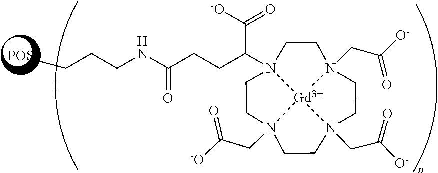

wherein POS is a matrix of polyorganosiloxane, and,
n is comprised between 4 and 200, preferably 4 and 80, and
wherein said nanoparticle has a mean hydrodynamic diameter comprised between 2 and 8 nm, preferably 2 and 6 nm.

An embodiment E46 of the present disclosure relates to the composition for its use according to any one of embodiments E39 to E44, wherein the radiation dose is between 20-30 Gy, for example about 25 Gy.

An embodiment E47 of the present disclosure relates to the composition for its use according to any one of embodiments E39 to E46, wherein said pharmaceutical composition comprising an efficient amount of said nanoparticles is injected intravenously at a concentration of at least 100 mg/mL, or at least 300 mg/mL, with a flow rate of between 2 and 3 mL/s at a dose of between 2 and 3 mL/kg.

An embodiment E48 of the present disclosure relates to the composition for its use according to any one of embodiments E39 to E47, wherein an image scan is acquired after 10 minutes, for example between 10 and 30 minutes after the injection of the nanoparticles to highlight the myocardial lesion.

An embodiment E49 of the present disclosure relates to the composition for its use according to any one of embodiments E39 to E48, wherein one or more image scans are acquired between 20 minutes and 24 hours after the injection of the nanoparticles.

An embodiment E50 of the present disclosure relates to the composition for its use according to any one of embodiments E39 to E49, wherein said cardiac disorder is scar of myocardial infarction.

An embodiment E51 of the present disclosure relates to the composition for its use according to any one of embodiments E39 to E49, wherein said cardiac disorder is arrhythmogenic fibrosis of cardiopathy.

An embodiment E52 of the present disclosure relates to the composition for its use according to any one of embodiments E39 to E49, wherein said cardiac disorder is a cardiac tumor.

An embodiment E53 of the present disclosure relates to the composition for its use according to any one of embodiments E39 to E49, for a preventive treatment for reperfusion lesion of myocardial infarction at the acute phase.

### BRIEF DESCRIPTION OF THE DRAWINGS

Other features, details and advantages will be shown in the following detailed description and on the figures, on which:

FIG. 1a. Conventional and K-edge images of 5 tubes containing AGuIX nanoparticles with increasing concentration of 0.5-1-2-4-8 mg/mL of gadolinium atoms (see number 5, 4, 3, 2, 1, respectively. The water tubes (number 6 to 8) as well as the material of the circular phantom are therefore not collected in K-edge imaging.

FIG. 1b. Contrast-to-noise ratios (CNR) measured in K-edge and conventional images. CNRs in K-edge imaging are higher than in conventional imaging due to a higher specificity.

FIG. 2. Longitudinal follow up of aortic opacification in conventional image showing a first pass phase 8 seconds after injection then a persistent second pass phase up to 3 minutes.

FIG. 3. Quantitative analysis of attenuation within the aorta in conventional image showing a first pass phase 8 seconds after injection then a persistent second pass phase up to 3 minutes.

FIG. 4a. First pass aortic phase SPCCT angiography in conventional image.

FIG. 4b. First pass aortic phase SPCCT angiography in conventional image (b1) in K-edge (b2).

FIG. 4c. First pass aortic phase SPCCT angiography in conventional image (c1) in K-edge (c2). (cross section of FIG. 4b).

FIG. 5. Paired comparison of aortic attenuations in aortic SPCCT angiographies in rabbits injected successively with Dotarem® (gadoteric acid, GUERBET, France) and AguIX 2 weeks apart before and after injection at 30 seconds, 1, 3 and 5 minutes.

FIG. 6. Aortic SPCCT angiography during first pass arterial phase using AguIX (A) versus Dotarem® (gadoteric acid, GUERBET, France) in an atherosclerotic rabbit model.

FIG. 7. Aortic SPCCT angiography during second pass arterial phase using AguIX (A) versus Dotarem® (gadoteric acid, GUERBET, France) in an atherosclerotic rabbit model. Conventional images using AguIX demonstrated a higher image quality than images with Dotarem®, with a clearer visualization of the aneurysmal aortic part (white head arrows) as well as for the inferior vena cava visualization (grey head arrows).

FIG. 8. Coronary SPCCT angiography after intra-arterial injection of 10 mL AguIX injected at 2 mL/s concentrated at 15 mg/mL of Gd atoms. Spectral material decomposition image into water and iodine (A) and spectral virtual 40 keV monochromatic image (B) show strong opacification of the left descending artery (LAD) and the circumflex artery (Cx).

FIG. 9. Coronary SPCCT angiography after intra-arterial injection of 10 mL AguIX injected at 2 mL/s concentrated at 15 mg/mL of Gd atoms. Conventional images (A) and K-edge images (B).

FIG. 10a. First pass arterial myocardial muscle SPCCT imaging after injection of 10 mL AguIX injected at 2 mL/s concentrated at 15 mg/mL of Gd atoms into the coronary left trunk with conventional (A) and K-edge images (B). Concentrations of gadolinium were measured of 4 mg/mL in the vascularized myocardium (white full star) and of 0 in the non-vascularized one (white empty star), while on conventional images.

FIG. 10b. Another First pass arterial myocardial muscle SPCCT imaging after injection of 10 mL AguIX injected at 2 mL/s concentrated at 15 mg/mL of Gd atoms into the coronary left trunk with conventional (bA) and K-edge images (bB). Concentrations of gadolinium were measured of 4 mg/mL in the vascularized myocardium (white full star) and of 0 in the non-vascularized one (white empty star), while on conventional images.

FIG. 11. Linear regression (A) and Bland Altman graphs (B, C) for comparison of infarct size between spectral 40 keV virtual monochromatic images and MRI and staining with triphenyltetrazolium chloride (TTC) in 7 pigs.

FIG. 12. Head-to-head comparison of MRI (first row), SPCCT (second row) and triphenyltetrazolium chloride (TTC) images for myocardial infarction imaging and size quantification on an ex vivo pig model.

FIG. 13. Comparison of Gd concentration measured with inductively coupled plasma spectrometry within infarcted and remote areas (a paired Walcoxon test was used).

FIG. 14. Multimodal ex vivo myocardial infarction imaging with 40 keV SPCCT monochromatic image (A), SPCCT material decomposition image (B) showing a hyperdensity in the infarcted zone concordant to the TTC staining images (infarction in white) (C). Concentration of gadolinium was measured of 1.34 mg/mL in the infarction area (white full star) while no signal was seen in the remote area (white empty star). Only infarcted myocardium (white full star) is seen in the spectral material decomposition image (B) due to its specificity, in comparison to monochromatic 40 keV image (A).

FIG. 15. Comparison of the difference in attenuation (Delta attenuation) between renal cortex and medulla as function of time and contrast medium used: top curve, Dotarem® (gadoteric acid, GUERBET, France); bottom curve, AguIX.

FIG. 16a. Kidney and urinary tract SPCCT imaging with AguIX (top row; A-C) compared with Dotarem® (gadoteric acid, GUERBET, France) (bottom row; D-F). Conventional images show high quality image for urinary tract and kidney imaging and demonstrate a better differentiation of the cortex (left arrows) at 8 seconds after injection, of the medulla (middle arrow) at 30 seconds and of the pelvic urinary tract at 5 minutes (right arrow).

FIG. 16b conventional (b1) and K-edge images b2) of kidney.

FIG. 16c conventional (cb1) and K-edge images c2) of bladder.

FIG. 16d conventional (d1) and K-edge images d2) of urinary tract.

FIG. 17a. Liver follow-up imaging performed after IV injection of AguIX (43 mg/mL Gd atoms, 2.5 mL/s), showing successive imaging of arteries (B arrows), veins (C and D arrows), and liver parenchyma (C and D stars) up to 1 minute.

FIG. 17b. Another liver SPCCT in conventional image (b1) in K-edge (b2).

FIG. 18a. Myocardial infarction in an ex vivo pig heart with 40 keV SPCCT virtual monochromatic image (A), spectral SPCCT material decomposition image (B), and T1-weighted sequence MRI imaging (C), in comparison with histological reference triphenyltetrazolium chloride (TTC) staining. SPCCT images are concordant with MRI and TTC, claiming the feasibility to identify myocardial infarction.

FIG. 18b. Another myocardial infarction in an ex vivo pig heart SPCCT conventional image (bA), K-edge image (bB), and superposition of bA and bB (bC).

FIG. 19. 3D T1-weighted MRI imaging 3 hours after injection of AguIX for aortic imaging (white arrows) after injection of 43 mg/mL Gd atoms at 2.5 mL/s. Satisfactory enhancement of the vena cava (grey arrow) is also noted.

FIG. 20. 3D liver T1-weighted MRI imaging 3 hours after injection of AguIX (concentrated at 43 mg/mL Gd atoms, injected at 2.5 mL/s) showing good visualization of the liver parenchyma (star 2), the portal and hepatic veins (grey arrows). Satisfactory enhancement of digestive walls (white empty arrow), kidney (star 3), spleen (star 4), heart (star 1) and vessels (white arrow) is also noted.

FIG. 21. 3D kidney T1-weighted MRI imaging 3 hours after injection of AguIX (concentrated at 43 mg/mL Gd atoms, injected at 2.5 mL/s) showing good visualization of the cortex (black full arrow); the medulla (black empty arrow) and urinary tract (white full arrow). Satisfactory enhancement of digestive walls (white empty arrow) is also noted.

FIG. 22. Conventional (1) and K-edge (2) abdominal imaging in rabbit.

### DETAILED DESCRIPTION

As mentioned above, the present disclosure relates to a method for imaging an anatomical structure in a subject in need thereof, comprising the following steps:
a) providing an injectable pharmaceutical composition comprising, as a contrast agent, at least one nanoparticle having a mean hydrodynamic diameter between 1 nm and 10 nm and comprising:
   a biocompatible matrix, such as polyorganosiloxane,
   at least one chelating agent covalently bonded to said biocompatible matrix,
   at least one element having a Z of at least 40 chelated to at least a part of the chelating agents,
b) injecting an effective amount of said pharmaceutical composition prepared at step a) to said subject, and,
c) acquiring an image scan of an anatomical structure of said subject by Spectral Photon Counting Computed Tomography (SPCCT) scanning.

As used herein, the term “anatomical structure” refers to any biological entity, more preferably of a human body, which occupies space and is distinguished from its surroundings. It can be a body part, including internal organs, tissues and organ systems. Examples of an anatomical structure for imaging according to the methods of the present disclosure includes without limitation the thorax, the abdomen, the pelvis, the brain, the heart, the arteriovenous system, the urinary tract and kidneys, and more generally any organs of a mammal, including without limitation, breast, lung, lymph nodes, bronchopulmonary area, or liver.

In step (b) of the method, “an effective amount” is an amount comprising sufficient nanoparticles as contrast agent for acquiring an image scan with SPCCT scanning.

As used herein, the term “SPCCT” (or Spectral Photon-Counting Computed Tomography, also referred as Photon-Counting computed tomography, PCCT), refers to a form of X-ray computed tomography (CT) in which X-rays are detected using a photon-counting detector. The first clinically-approved PCCT system was cleared by the Food and Drug Administration (FDA) in September 2021. SPCCT device employs a photon-counting detector (PCD) which registers the interactions of individual photons. By keeping track of the deposited energy in each interaction, the detector pixels of a PCD each record an approximate energy spectrum, making it a spectral or energy-resolved CT technique. SPCCT scan can be performed on every region of the body, e.g. a human body, depending on the desired application. An example of imaging SPCCT device is Siemens NAEOTOM Alpha.

### The Nanopartlcles for Use as Contrast Agent

The nanoparticles for use as contrast agents in the imaging methods as disclosed herein have a mean hydrodynamic diameter between 2 nm and 8 nm and comprises:
a biocompatible matrix forming the nanoparticle,
at least one chelating agent covalently bonded to said biocompatible matrix,
at least one element having a Z of at least 40 chelated to at least a part of the chelating agents,

The size distribution of the nanoparticles is, for example, measured using a commercial particle sizer, such as a Malvern Zetasizer Nano-S particle sizer based on PCS (Photon Correlation Spectroscopy). For the purposes of the invention, the term “mean hydrodynamic diameter” or “mean diameter” is intended to the harmonic mean of the diameters of the particles. A method for measuring this parameter is also described in standard ISO 13321:1996.

The small size of the nanoparticles, between 1 and 10 nm, enables rapid renal elimination after injection. In specific embodiments, the nanoparticles have a mean hydrodynamic diameter between 2 and 8 nm, preferably between 2 and 6 nm.

In particular embodiments, the nanoparticles are chosen such that they have a relaxivity r1 per particle of between 50 and 5000 mM⁻¹·s⁻¹ (at 37° C. and 1.4 T) and/or a Gd weight ratio of at least 5%, for example between 5% and 30%.

Suitable biocompatible matrix includes without limitation biocompatible polymers, such as polyethylene glycol, polyethyleneoxide, polyacrylamide, biopolymers, polysaccharides, or polysiloxane. In specific embodiments, the nanoparticle comprises a silica-based matrix, for example essentially consisting of polyorganosiloxane.

The nanoparticles for use in the imaging methods comprise chelating agents complexed to metal ions, wherein the chelating agents are covalently bonded to the biocompatible matrix, e.g. polyorganosiloxane.

As used herein, the term “chelating agent” refers to a group capable of complexing one or more metal ions.

Exemplary chelating agents include, but not limited to, 1,4,7,10-tetraazacyclododecane-1,4,7,10-tetraacetic acid (DOTA), diethylene triaminepentaacetic acid (DTPA), ethylene diamine tetra-acetic acid (EDTA), ethyleneglycol-0,0′-bis(2-aminoethy)-N,N,N′,N′-tetraacetic acid (EGTA), 1,2-bis(o-aminophenoxy)ethane-N,N,N′,N′-tetraacetic acid (BAPTA), 1,4,7-triazacyclononanetriacetic acid (NOTA), 1,4,7,10-tetraazacycododececane, 1-(glutaric acid)-4,7,10-triacetic acid (DOTAGA), deferoxamine (DFO), (1,4,7,10-tetrakis(carbamoylmethy)-1,4,7,10 tetraazacyclododecane) (DOTAM) and 2-(bis(2-(2,6-dioxomorpholino)ethyl)amino) acetic acid DTPABA.

In some embodiments, said chelating agent is selected among the following:

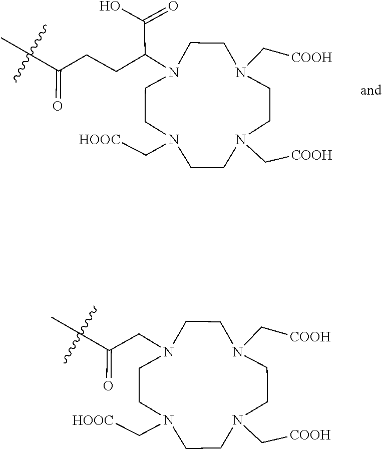 [SMILES: *C(=O)CCC(C(=O)O)N1CCN(CC(=O)O)CCN(CC(=O)O)CCN(CC(=O)O)CC1 ; *C(=O)CN1CCN(CC(=O)O)CCN(CC(=O)O)CCN(CC(=O)O)CC1]

wherein the wavy bond indicates the bond connecting the chelating agent to a linking group of a biocompatible matrix forming the nanoparticle.

A portion or all of the chelating agents in the nanoparticles are complexed with a high-Z element, which high-Z element provides contrast agent properties to the nanoparticles for SPCCT scanning, and, optionally, other imaging methods, such as magnetic resonance imaging (MRI). The high-Z element is therefore advantageously selected among the metal ion which have contrast agent properties.

Said high-Z element as used herein is an element with an atomic Z number higher than 40, for example higher than 50.

In specific embodiments, said high-Z element is selected in the group consisting of lanthanum, cerium, praseodymium, neodymium, promethium, samarium, europium, gadolinium, terbium, dysprosium, holmium, erbium, thulium, ytterbium, lutetium, hafnium, tantalum, tungsten, rhenium, osmium, iridium, platinum, gold and bismuth, preferably in the group consisting of gadolinium and bismuth.

The high-Z elements are preferably cationic elements, comprised as complexes with chelating agents, such as organic chelating agents.

In specific embodiments, at least 65% of the chelating agent present in the nanoparticle is chelated to at least one high-Z element, preferably at least 75% and more preferably at least 80%.

In a specific embodiment, that may be preferably combined with the previous embodiment, said chelating agent is complexed to a metal ion, and more specifically to gadolinium and/or bismuth, preferably DOTA or DOTAGA chelating Gd³⁺ and/or Bi³⁺.

In specific and preferred embodiments, the mean number of high-Z element per nanoparticle, for example the number of rare earth elements, e.g. gadolinium (optionally as chelated with DOTAGA) per nanoparticle, is between 4 and 200, preferably between 4 and 80, typically around 15.

In certain embodiments, the method of imaging as disclosed herein is carried out with a nanoparticle which comprises or essentially consists of:
polyorganosiloxane with a silicon weight ratio of at least 8% of the total weight of the nanoparticle, preferably between 8% and 50%,
chelating agents covalently bound to said polyorganosiloxane, in a proportion comprising between 4 and 200, preferably between 4 and 80 per nanoparticle, and,
at least one element having a Z of at least 40 chelated to at least a part of the chelating agents.

In a specific embodiment, said nanoparticles are gadolinium-chelated polyorganosiloxane nanoparticles of the following formula:

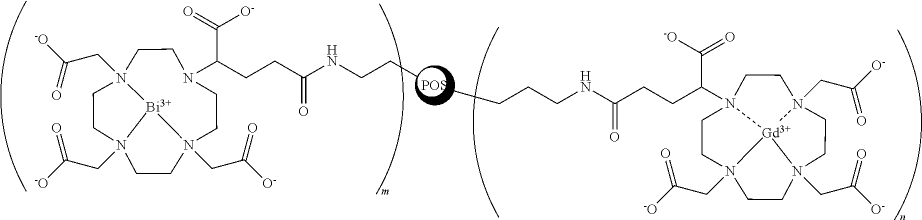

wherein POS is a matrix of polyorganosiloxane, and,
n is comprised between 0 and 100,
m is comprised between 0 and 100,
provided that n+m is comprised between 4 and 200, preferably 4 and 80, and
wherein said nanoparticle has a mean hydrodynamic diameter comprised between 2 and 8 nm, preferably 2 and 6 nm.

In another specific embodiment, said nanoparticles are gadolinium-chelated polyorganosiloxane nanoparticles of the following formula:

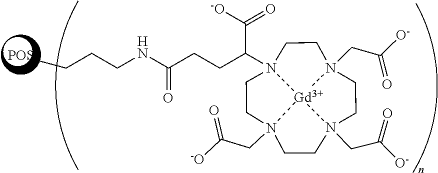

wherein POS is a matrix of polyorganosiloxane, and,
n is comprised between 4 and 200, preferably 4 and 80, and
wherein said nanoparticles have a mean hydrodynamic diameter comprised between 2 and 8 nm, preferably between 2 and 6 nm.

More specifically, said gadolinium-chelated polysiloxane nanoparticles as described in the above formula are ultrafine nanoparticles, more preferably AguIX nanoparticles as described in the next section.

Ultrafine nanoparticles that can be used according to the methods of the disclosure may be obtained or obtainable by a top-down synthesis route comprising the steps of:
a. obtaining a metal (M) oxide core, wherein M is a high-Z element as described previously, preferably gadolinium,
b. adding a polyorganosiloxane shell around the M oxide core, for example via a sol gel process,
c. grafting a chelating agent to the polyorganosiloxane shell, so that the chelating agent is bound to said polyorganosiloxane shell by an —Si—C— covalent bond, thereby obtaining a core-shell precursor nanoparticle, and,
d. transferring the core-shell precursor nanoparticle in an aqueous solution for dissolution of the metal oxide core and purifying,
   wherein the grafted agent is in sufficient amount to dissolve the metal (M) oxide core at step d. and to complex the cationic form of (M) thereby reducing the mean hydrodynamic diameter of the resulting hybrid nanoparticle to a mean hydrodynamic diameter less than 10 nm, for example, between 2 and 8 nm, typically between 2 and 6 nm.

These nanoparticles obtained according to the mode described above do not comprise a core of metal oxide encapsulated by at least one coating. More details regarding the synthesis of these nanoparticles are given hereafter.

This top-down synthesis method results in observed sizes typically of between 2 and 8 nm, more specifically between 2 and 6 nm. The term then used herein is ultrafine nanoparticles.

Alternatively, another “one-pot” synthesis method is described hereafter to prepare said core-free nanoparticles with a mean hydrodynamic diameter less than 10 nm, for example, between 2 and 8 nm, typically between 2 and 6 nm.

Further details regarding these ultrafine or core-free nanoparticles, the processes for synthesizing them and their uses are described in patent application WO2011/135101, WO2018/224884 or WO2019/008040, which is incorporated by way of reference.

Generally, those skilled in the art will be able to easily produce nanoparticles used according to the invention. More specifically, the following elements will be noted:

For nanoparticles of core-shell type, based on a core of lanthanide oxide or oxyhydroxide, use may be made of a production process using an alcohol as solvent, as described for example in P. Perriat et al., J. Coll. Int. Sci, 2004, 273, 191; O. Tillement et al., J. Am. Chem. Soc., 2007, 129, 5076 and P. Perriat et al., J. Phys. Chem. C, 2009, 113, 4038.

For the POS matrix, several techniques can be used, derived from those initiated by Stoeber (Stoeber, W; J. Colloid Interf Sci 1968, 26, 62). Use may also be made of the process used for coating as described in Louis et al. (Louis et al., 2005, Chemistry of Materials, 17, 1673-1682) or international application WO 2005/088314.

In practice, synthesis of ultrafine nanoparticles is for example described in Mignot et al. Chem. Eur. J. 2013, 19, 6122-6136: Typically, a precursor nanoparticle of core/shell type is formed with a lanthanide oxide core (via the modified polyol route) and a polyorganosiloxane shell (via sol/gel); this object has, for example, a mean hydrodynamic diameter of around 5-10 nm. A lanthanide oxide core of very small size (adjustable less than 10 nm) can thus be produced in an alcohol by means of one of the processes described in the following publications: P. Perriat et al., J. Coll. Int. Sci, 2004, 273, 191; O. Tillement et al., J. Am. Chem. Soc., 2007, 129, 5076 and P. Perriat et al., J. Phys. Chem. C, 2009, 113, 4038.

These cores can be coated with a layer of polyorganosiloxane according to, for example, a protocol described in the following publications: C. Louis et al., Chem. Mat., 2005, 17, 1673 and O. Tillement et al., J. Am. Chem. Soc., 2007, 129, 5076.

Chelating agents specific for the intended metal cations (for example DOTAGA for Gd³⁺) are grafted to the surface of the polyorganosiloxane; it is also possible to insert a part thereof inside the layer, but the control of the formation of the polyorganosiloxane is complex and simple external grafting gives, at these very small sizes, a sufficient proportion of grafting.

The nanoparticles may be separated from the synthesis residues by means of a method of dialysis or of tangential filtration, for example on a membrane comprising pores of appropriate size.

The core is destroyed by dissolution (for example by modifying the pH or by introducing complexing molecules into the solution). This destruction of the core then allows a diffusion and a rearrangement of the polyorganosiloxane layer (according to a mechanism of slow corrosion or collapse), which makes it possible to finally obtain a polyorganosiloxane object with a complex morphology, the characteristic dimensions of which are of the order of magnitude of the thickness of the polyorganosiloxane layer, i.e. much smaller than the objects produced up until now.

Removing the core thus makes it possible to decrease from a particle size of approximately 5-10 nanometers in mean hydrodynamic diameter to a size below 8 nm, for example between 2-8 nm. Furthermore, this operation makes it possible to increase the number of M (e.g. gadolinium) per nm³ in comparison with a theoretical polyorganosiloxane nanoparticle of the same size but comprising M (e.g. gadolinium) only at the surface. The mean number of M per nanoparticle can be evaluated by virtue of the M/Si atomic ratio measured by EDX or by ICP/MS. Typically, this number of M per ultrafine nanoparticle may be comprised between 4 and 200, preferably 4 and 80.

In one specific embodiment, the nanoparticles according to the disclosure each comprises a chelating agent which has an acid function, for example DOTA or DOTAGA. The acid function of the nanoparticle is activated for example using EDC/NHS (1-ethyl-3-(3-dimethylaminopropy)carbodiimide/N-hydrosuccinimide) in the presence of an appropriate amount of targeting molecules. The nanoparticles thus grafted are then purified, for example by tangential filtration.

Alternatively, the nanoparticles according to the present disclosure may be obtained or obtainable by a synthesis method (one-pot synthesis method) comprising the mixing of at least one hydroxysilane or alkoxysilane which is negatively charged at physiological pH and at least one chelating agent chosen from polyamino polycarboxylic acids with
at least one hydroxysilane or alkoxysilane which is neutral at physiological pH, and/or
at least one hydroxysilane or alkoxysilane which is positively charged at physiological pH and comprises an amino function,
   wherein:
the molar ratio A of neutral silane(s) to negatively charged silane(s) is defined as follows: 0≤A≤6, preferably 0.5≤A≤2;
the molar ratio B of positively charged silane(s) to negatively charged silane(s) is defined as follows: 0≤B≤5, preferably 0.25≤B≤3;
the molar ratio C of neutral and positively charged silanes to negatively charged silane(s) is defined as follows 0<C≤8, preferably 1≤C≤4.

According to a more specific embodiment of such one pot synthesis method, the method comprises the mixing of at least one alkoxysilane which is negatively charged at physiological pH, said alkoxysilane being chosen among APTES-DOTAGA, TANED, CEST and mixtures thereof, with
at least alkoxysilane which is neutral at physiological pH, said alkoxysilane being chosen among TMOS, TEOS and mixtures thereof, and/or
APTES which is positively charged at physiological pH,
   wherein:
the molar ratio A of neutral silane(s) to negatively charged silane(s) is defined as follows: 0≤A≤6, preferably 0.5≤A≤2;
the molar ratio B of positively charged silane(s) to negatively charged silane(s) is defined as follows: 0≤B≤5, preferably 0.25≤B≤3;
the molar ratio C of neutral and positively charged silanes to negatively charged silane(s) is defined as follows 0<C≤8, preferably 1≤C≤4.

According to a specific embodiment, the one-pot synthesis method comprises the mixing of APTES-DOTAGA which is negatively charged at physiological pH with
at least one alkoxysilane which is neutral at physiological pH, said alkoxysilane being chosen among TMOS, TEOS and mixtures thereof, and/or
APTES which is positively charged at physiological pH,
   wherein:
the molar ratio A of neutral silane(s) to negatively charged silane(s) is defined as follows: 0≤A≤6, preferably 0.5≤A≤2;
the molar ratio B of positively charged silane(s) to negatively charged silane(s) is defined as follows: 0≤B≤5, preferably 0.25≤B≤3;
the molar ratio C of neutral and positively charged silanes to negatively charged silane(s) is defined as follows 0<C≤8, preferably 1≤C≤4.

### AGuIX Nanopaticles

In a more particularly preferred embodiment, said gadolinium-chelated polyorganosiloxane based nanoparticle is the AGuIX nanoparticle of the formula below

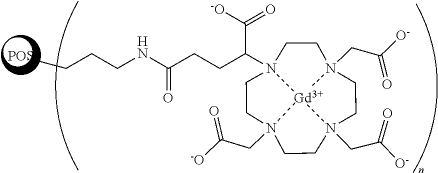

wherein POS is polyorganosiloxane and n is between 4 and 200, preferably between 4 and 80, and having a mean hydrodynamic diameter of 5±3 nm and a mean mass of between 5 and 250 kDa, preferably between 5 and 100 kDa.

In specific embodiments, said AGuIX nanoparticle can also be described by the average chemical formula: (GdSi₃₋₈C₂₄₋₃₄N—O₁₅₋₃₀H₄₀₋₆₀, 5-15H₂O)ₙ, where n is between 4 and 200, preferably between 4 and 80.

Methods for preparing AGuIX nanoparticles is described for example in G. Le Duc, S. Roux, A. Paruta-Tuarez, S. Dufort, E. Brauer, A. Marais, C. Truillet, L. Sancey, P. Perriat, F. Lux, O. Tillement, «Advantages of gadolinium based ultrasmall nanoparticles vs molecular gadolinium chelates for radiotherapy guided by MRI for glioma treatment», Cancer Nanotechnology, 2014, 5, 4.

### The Pharmaceutical Composition

The composition comprising said nanoparticles for use as provided herein is preferably administered in the form of an injectable pharmaceutical formulation of nanoparticles. These formulations can be prepared as described herein or elsewhere, and can be administered by a variety of routes.

In a particular embodiment, said pharmaceutical composition for use as described herein, contain, as the contrast agent, a suspension of nanoparticles, as provided herein, in combination with one or more pharmaceutically acceptable carriers (excipients).

In a specific embodiment, said pharmaceutical composition, is prepared as a sterile lyophilized powder, contained in a pre-filled vial to be reconstituted, for example in an aqueous solution for intravenous injection.

Such powder may further contain one or more additional excipients, and in particular CaCl₂).

Said lyophilized powder may be reconstituted in an aqueous solution, typically water for injection. Accordingly, in specific embodiments, said pharmaceutical composition is a solution for injection, comprising, as a contrast agent, an efficient amount of said high-Z containing nanoparticles, typically gadolinium-chelated polyorganosiloxane based nanoparticles, and more specifically AGuIX nanoparticles as described herein.

In another specific embodiment, said pharmaceutical composition is an aqueous composition for injection, for example contained in a vial, comprising, as a contrast agent, an efficient amount of said high-Z containing nanoparticles, typically gadolinium-chelated polyorganosiloxane based nanoparticles, and more specifically AGuIX nanoparticles as described herein.

In certain specific embodiments, said pharmaceutical composition, typically the aqueous composition or composition for injection comprises said high-Z containing nanoparticles, typically gadolinium-chelated polyorganosiloxane based nanoparticles, and more specifically AGuIX nanoparticles at a concentration between 50 and 500 mg/mL, typically between 100 and 350 mg/mL.

### The Methods of the Present Disclosure

The method of imaging of the present disclosure comprises at least the following steps
a) injecting an effective amount of said pharmaceutical composition comprising the nanoparticles as a contrast agent for SPCTT imaging of the anatomical structure of said subject (the injecting step), and
b) acquiring an imaging scan of said anatomical structure by SPCCT scanning (the imaging step).

The pharmaceutical composition for use at the injecting step and their nanoparticles have been described in details in the previous sections. In preferred embodiments of the methods, said pharmaceutical composition comprises gadolinium containing nanoparticles, and more preferably AGuIX Gd-containing nanoparticles.

In particular, the inventors have shown that pharmaceutical composition comprising gadolinium containing nanoparticles such as AGuIX Gd-containing nanoparticles provides remarkable contrast agent properties for SPCCT scanning, and may therefore advantageously be used for certain diagnostic and theranostic applications.

In specific embodiments, the pharmaceutical agent is administered at the injecting step via intravenous route (central or peripheric), intra-arterial route, aortic route, intra-coronary arteries, intra-cerebral arteries, intra-vesical, intra-tumoral, intra-peritoneal, intra-pleural, intra-articular, in the bone, in the small and large intestine, in the stomach.

The skilled person will adapt the concentration of the nanoparticles in the injectable composition, the debit of injection and the volume depending on the use, the route of administration and the patient in need thereof.

In particular embodiments, the method is carried out with said nanoparticles (preferably AGuIX nanoparticles) comprised in an injectable composition at a concentration between 50 and 500 mg/mL, preferably between 80 and 400 mg/mL and more preferably between 100 and 350 mg/mL.

In particular embodiments, the injectable pharmaceutical composition is administered at a flow rate of about 2 to 15 mL/s for a volume adapted to the weight of the patient and the application. Volumes of composition may vary for example from 10-200 mL for a concentration of nanoparticles between 100 and 300 mg/mL. More specific examples of flow rate, concentration and volume of composition are disclosed hereafter for preferred use, as well in the experimental section.

In other particular embodiments, the method is carried out with said nanoparticle comprised in an injectable composition with a concentration of chelated elements having a Z of at least 40, typically gadolinium, between 2.5 and 150 mg/mL, preferably between 4 and 120 mg/mL and more preferably between 5 and 105 mg/mL.

In specific embodiments, said nanoparticles are administered as the sole contrast agent. In other specific embodiments, one or more additional contrast agents is(are) administered, concomitantly or sequentially with the nanoparticles as disclosed herein, typically AGuIX nanoparticles. In particular, it is possible to take advantage of K-edge imaging to differentiate images from different contrast agents. In specific embodiments, iodine or iodinated contrast agent is administered as a second contrast agent. In other specific embodiments, nanoparticles with gadolinium are administered as a first contrast agent in combination (concomitantly or sequentially) with nanoparticles with another element as a second contrast agent (for example, dysprosium, holmium, erbium, thulium, ytterbium, lutetium, hafnium, tantalum, tungsten, rhenium, osmium, iridium, platinum, gold and bismuth).

As used herein, the term “image scan” refers to the acquisition with one scan of SPCCT device of digital image data of at least an anatomical structure of a subject.

The skilled person will also determine the time of acquisition of the image scan after the injection of the contrast agent, depending on the applications.

In a specific embodiment, at least one image scan is acquired between 5 minutes and 10 minutes after injection of the contrast agent (i.e. said injectable pharmaceutical composition comprising nanoparticles as described above). In other specific methods, more than one image scan is acquired after injection of the contrast agent (serial acquisitions). For example, an image scan may be acquired at early stages, typically 15 seconds, 30 seconds, 1, 2, 3, 4, 5, 6, 7, 9, and/or 10 minutes, and/or at later stages, typically 15 minutes, 30 minutes, 45 minutes, 1 hour, 2 hours, 3 hours, 4, 5, 6, 7, 8, 9, 10, 11, 12, 13, 14, 15, 16, 17, 18, 19, 20, 21, 22, 23 hours and/or 1, 2, 3, 4, 5, 6, 7 days after injection.

Methods for acquiring image scan using a SPCCT device are for example described in Wllemink et al., Radiology 2018; 289:293-312 (doi.org/10.1148/radiol.2018172656) or in Si-Mohamed et al., Nuclear Instruments and Methods in Physics Research Section A: Accelerators, Spectrometers, Detectors and Associated Equipment, volume 873, 21 Nov. 2017, Pages 27-35 (doi.org/10.1016/j.nima.2017.04.014).

Methods for acquiring a dynamic K-edge angiography in vivo in rabbits in presence of one or two contrast agents such iodine contrast agent and gold nanoparticles is provided in Cormode and Si-Mohamed et al (doi: 10.1038/s41598-017-04659-9) as well as in Si-Mohamed et al. (doi: 10.1148/radiol.2021203968).

Methods for acquiring longitudinal molecular K-edge imaging for bone marrow, liver, spleen and lymph nodes in presence of gold nanoparticles is provided in Si-Mohamed et al (doi: 10.1039/c7nr01153a).

Methods for acquiring one or two K-edge contrast agents simultaneously is provided in Si-Mohamed et al. (doi: 10.1186/s41747-018-0063-4).

Images are then reconstructed for visualization using for example conventional protocols. Methods of images reconstruction are for example described in the above-mentioned articles. In specific embodiments, images are reconstructed to achieve conventional, spectral, monochromatic images at low and high energy between 40 and 200 keV and/or spectral material decomposition into 2 non-K-edge materials (e.g., water-iodine, uric acid-calcium, photoelectric-Compton), and/or spectral material decomposition into a chosen K-edge material.

### Specific Applications of the Method of Imaging

The method generally described in the previous section can be applied for preferred applications which are further described below in detail as well as in the Examples.

The methods as described herein are particularly useful for assisting in diagnosis or monitoring specific conditions.

In embodiment, the condition is selected from the group consisting of cardiac ischemia, myocardial infarction, and stroke.

In another embodiment, the condition is selected from the group consisting of vascular calcification, coronary stenosis or in-stent restenosis.

In an embodiment, said condition is acute pulmonary embolism.

In another embodiment, the method is also useful for diagnosing primary or secondary pulmonary tumors, viral pneumopathies, interstitial fibrosis, chronic obstructive pulmonary disorders, cystic fibrosis, and bronchiolitis, wherein said nanoparticles, typically gadolinium chelated nanoparticles, preferably AGuIX nanoparticles, is administered via the airways.

In another embodiment, the method is useful for diagnosing primary or secondary bladder tumor, primary or secondary ureteral tumor primary or secondary renal tumor, lithiasis or renal insufficiency, wherein said nanoparticle typically gadolinium chelated nanoparticles, preferably AGuIX nanoparticles, is administered via urinary tract route.

In an embodiment, said condition is a cancer, for example for detecting a solid tumor, preferably selected from the group consisting of glioblastoma, brain metastases, meningioma, or primary tumor of uterine cervix, rectum, lung, head and neck, prostate, colorectal, liver, and pancreas cancers. More preferably, said tumor is a brain metastases, typically a brain metastases from melanoma, lung, breast, kidney primary cancers. In such embodiment, said nanoparticles, may also be used as a radiosensiizing agent for treating said tumor by radiotherapy. In a specific embodiment, said nanoparticles is administered via intratumoral route.

### Methods of Imaging of Thoraco-Abdominopelvian and Cerebral Anatomic Structure

A particular advantageous use of the above methods of imaging relates to methods of imaging of thoracic, abdominal, pelvic and cerebral anatomical structure or organs, in particular for diagnostic and monitoring of arteriovenous pathologies, oncologic, inflammatory disorders and traumatic disorders.

For such use, said injectable pharmaceutical composition comprising the nanoparticles as above-defined, preferably gadolinium chelated nanoparticles, and more preferably AGuIX nanoparticles is administered via intravenous route, for example at a concentration between 150 and 400 mg/mL, typically 300 mg/mL, and with flow rate of 2 to 5 mL/s, typically 2.5 mL/s. The volume will be adapted to the patient and may be for example comprised between 2 and 4 mL/kg, typically 2.5 mL/kg.

The image is first acquired at an early stage, for example, at 10-20 seconds after the end of the injection of the contrast agent for an arterial phase imaging, and then at a later stage, for example between 45 and 120 seconds, typically 60 seconds, for arteriovenous imaging and enhancement of the thoracic, abdominal and pelvic organs. A late phase scan acquisition may be performed 3, 4, 5 minutes or later after injection for evaluating the wash out in the organs and also for taking benefit of the delayed time imaging window into the blood compartment.

For oncologic imaging, late scan acquisitions at 30 minutes, 40 minutes, 1 hour, 2 hours, 3 hours, 4, 5, 6, 7, 8, 9, 10, 11, 12, 13, 14, 15, 16, 17, 18, 19, 20, 21, 22, 23 hours, and/or up to one day are performed for following the enhancement kinetics and for performing a post-operative or post-radiotherapy imaging. Post-operative or post-radiotherapy imaging may thus be performed without the need to re-inject due to the extended time imaging window.

The images are then reconstructed by (i) conventional spectral monochromatic images at low energy, in order to benefit of the boost in attenuation of the gadolinium nanoparticles and/or (ii) spectral k-edge of gadolinium at 50.2 keV to obtain specific and quantitative imaging of the tissues.

### Methods of Cardiac Imaging

Another advantageous use of the methods of the present disclosure is for cardiac imaging in a mammal, typically a human subject.

For such use, the pharmaceutical composition containing the nanoparticles as contrast agent (preferably AGuIX nanoparticles) is injected intra-arterially in a subject. In specific embodiments, said subject is suffering from coronary artery disease or acute chest pain or acute coronary syndrome.

About 100 to 500 mg/mL of said nanoparticles as disclosed herein (e.g. AGuIX, typically 100-300 mg/mL of AGuIX) is injected in the coronary arteries or in the aortic root during an invasive coronary angiography, via a catheter, e.g. at a flow rate between 5 and 15 mL/s, of a volume independent from the patient weight.

The volume is adjusted to the distribution volume inside the coronary arteries or the aortic root. The amount of the nanoparticles is adjusted to enable a coronary angiogram.

A short-term imaging (e.g. between 5 to 60 minutes after injection) of the myocardial muscle in the SPCCT room is performed for evaluating the myocardial damages.

In specific embodiments, the subject may undergo a cardiac magnetic resonance imaging (MRI) at mid-term for a complementary baseline evaluation of the infarct size.

The reconstruction protocol typically consists in reconstructing conventional images, spectral virtual monochromatic images (for example 40 keV) for increasing the attenuation of the nanoparticles, and spectral K-edge when gadolinium is used as high-Z element for enabling a specific and quantitative tissue imaging.

In specific embodiments, said cardiac imaging method as described herein is performed in methods for diagnosis of the size and involvement of reperfusion lesion occurring during the acute phase of a myocardial infarction. In other specific embodiments, it is performed for diagnosis and/or monitoring of the size of myocarditis. In other specific embodiments, it is performed for diagnosis and/or monitoring of aortic lesions, typically after an invasive coronary angiography (ulcer, aortic dissection, intramural hematoma).

### Brain Imaging

Another advantageous use of the methods of the present disclosure is for brain imaging in a mammal, typically a human subject.

For such use, the pharmaceutical composition containing the nanoparticles as contrast agent (preferably AGuIX nanoparticles) is injected into cerebral arteries, typically during an endovascular mechanical thrombectomy.

About 100 to 500 mg/mL of nanoparticles as disclosed herein (e.g. AGuIX, typically 100-300 mg/mL of AGuIX) is injected in the cerebral arteries during an invasive angiography, via a catheter, at a flow rate between 5 and 15 mL/s, of a volume independent from the patient weight. The volume is adjusted to the distribution volume inside the coronary arteries or the aortic root. Injection of the nanoparticles enables a cerebral angiogram.

A short imaging of the stroke in the SPCCT room is then performed (e.g. between 0 to 60 minutes after injection) for evaluating the brain damages, and for differentiating the necrosis from the hemorrhage (Brain Commun. 2020 Nov. 11; 2(2):fcaa193.doi: 10.1093/braincomms/fcaa193).

In specific embodiments, the patient may further undergo a brain MRI at mid-term for a baseline evaluation of the infarct size.

In specific embodiments, said methods of brain imaging as described herein is performed for diagnosis and/or monitoring of stroke size and involvement of hemorrhage within the necrosis. In other embodiments, said methods of brain imaging is performed for diagnosis and monitoring of blood brain barrier permeability after mechanical thrombectomy for stroke, inflammatory, infectious and tumoral lesions in the brain.

Cardiovascular Imaging with Extended Imaging Time Window in Human

Another advantageous use of the methods of the present disclosure is for cardiovascular imaging in a mammal, typically a human subject. As compared to other imaging methods, the methods benefit of the long-time acquisition of the images and high resolution of the images for an improved cardiovascular imaging.

In one specific embodiment, the pharmaceutical composition containing the nanoparticles as contrast agent (preferably gadolinium containing nanoparticles, and more preferably AGuIX nanoparticles) is injected through intravenous injection in subject in need thereof. About 200 to 400 mg/mL of said nanoparticles as disclosed herein (e.g. AGuIX, typically 300 mg/mL of AGuIX) is injected intravenously, via peripheral or central catheter, at a flow rate e.g. between 2 and 6 mL/s, for example 2.5 mL/s, of a volume adjusted to the patient weight (typically between 1 to 3 mL/kg, for example between 1.5 to 2.5 mL/kg). Several images are acquired sequentially right immediately following the end of the injection (for example between 10 and 30 seconds) and up to 3 minutes, and for example, 2 to 5 scan acquisitions are performed, from 10 seconds to an extended time comprised between 3 and 5 minutes, for an imaging of the arteries. In specific embodiments, imaging is obtained of arteries selected from coronary, neck and cerebral arteries, and/or for an imaging of the aorta and its branches. In specific embodiments, the coronary arteries are acquired via an ECG-gated acquisition.

In another specific embodiment, the pharmaceutical composition containing the nanoparticles as contrast agent (preferably AGuIX nanoparticles) is injected through the artery in subject in need thereof. About 100 to 300 mg/mL of said nanoparticles as disclosed herein (e.g. AGuIX, typically 100-300 mg/mL of AGuIX) is injected in the coronary arteries or the aortic root, via a catheter localized in the artery of interest, at a flow rate between 2 and 15 mL/s, of a volume independent of the patient weight but adjusted to the distribution volume of the patient of interest. Several image scans are acquired sequentially right immediately following the injection (for example between 0 and 10 seconds) and up to 5 minutes, and for example, 2 to 5, 6, 7, 8, 9, 10 scan acquisitions are performed from 15 seconds to an extended time comprised between 3 and 10 minutes, typically between 3 and 5 minutes, thereby benefiting of the extended time imaging window.

The reconstruction protocol typically consists in reconstructing conventional images, spectral virtual monochromatic images (for example 40 keV) for increasing the attenuation of the nanoparticles (e.g. Gd chelated nanoparticles and more specifically AGuIX) and spectral K-edge when gadolinium is used as high-Z element, presenting with a K-edge at 50.2 keV for enabling a specific and quantitative tissue imaging.

Such method of cardiovascular imaging may be performed for diagnosis and/or monitoring of coronary artery disease, neck and brain arteries diseases, aortic and its branches (medullar artery, bronchial and kidney arteries for example) diseases.

### Urinary Tract and Kidney Imaging

Another advantageous use of the methods of the present disclosure is for urinary tract and kidney imaging in a mammal, typically a human subject.

In one specific embodiment, the pharmaceutical composition containing the nanoparticles as contrast agent (preferably gadolinium containing nanoparticles, and more preferably AGuIX nanoparticles) is injected through intravenous injection in a subject in need thereof. About 200 to 400 mg/mL of said nanoparticles as disclosed herein (e.g. AGuIX, typically 300 mg/mL of AGuIX) is injected intravenously, via peripheral or central catheter, at a flow rate between 2 and 5 mL/s, for example 2.5 mL/s, of a volume adjusted to the patient weight (typically between 1 to 3 mL/kg, for example between 1.5 to 2.5 mL/kg). Several images are acquired sequentially, with one image scan right immediately following the end of the injection, e.g. between 10 and 20 seconds, typically 15 seconds, and several image acquisitions are performed from at least 3 minutes to up to 3 hours for a urinary tract imaging.

In another specific embodiment, the pharmaceutical composition containing the nanoparticles as contrast agent (preferably gadolinium containing nanoparticles, and more preferably AGuIX nanoparticles) is injected in the urinary tract, via a nephrostomy or a bladder catheter. About 100 to 300 mg/mL of said nanoparticles as disclosed herein (e.g. AGuIX, typically 100-300 mg/mL of AGuIX) is injected, at a flow rate between 1 and 5 mL/s, of a volume independent of the patient weight but adjusted to the distribution volume of the urinary tract of the patient (between 50-1000 mL). Several image scans are acquired sequentially right immediately following the injection and up to 360 minutes.

The reconstruction protocol typically consists in reconstructing conventional images, spectral virtual monochromatic images (for example 40 keV) for increasing the attenuation of the nanoparticles (e.g. Gd chelated nanoparticles and more specifically AGuIX) and spectral K-edge when gadolinium is used as high-Z element, presenting with a K-edge at 50.2 keV for enabling a specific and quantitative tissue imaging. In specific embodiments, K-edge imaging allows the differentiation between the cavities filled with the nanoparticles (e.g. AGuIX) and the spontaneous hyperdensities (e.g. calcification) for a better depiction of kidney stones.

Such method of urinary tract and kidney imaging may be performed in methods for diagnosis and monitoring of kidney stone disease, urinary tract and kidney tumors, chronic renal failure by means of quantification of the glomerular filtration rate, which quantification may be performed as disclosed for example in Radiology. 2011 August; 260(2):414-20. doi: 10.1148/radiol.11101317.

### Organ Imaging

Another advantageous use of the methods of the present disclosure is for organ imaging in a mammal, typically a human subject, in particular for the detection of tumors in such organ.

For such use, the pharmaceutical composition containing the nanoparticles as contrast agent (preferably AGuIX nanoparticles) is injected in situ in the organ of interest, for example selected from the group consisting of lymph nodes, breast, bronchopulmonary system, liver or cardiovascular system.

In specific embodiments, about 100 to 500 mg/mL of nanoparticles as disclosed herein (e.g. AGuIX, typically 100-300 mg/mL of AGuIX) is injected in situ of different organs, for example at flow rate of 1-5 mL/s, with a volume adjusted to distribution volume of each organ (i.e. 10-20 mL for lymphatic system, 50-1000 mL for urinary tract, 5-10 mL for brain ventricular system, 1-10 mL for lymph nodes, 1-20 mL for breast, bronchopulmonary, liver and vascular system).

Scan acquisitions are performed at the end of injection in order to show a local deposition or peripheral distribution in sentinel lesions such as lymph nodes in breast cancer. Time points for imaging can be repeated and extended from hours, typically after 1 hour, 2, 3, 4, 5, 6, 7, 8, 9, 10, 11, 12 hours or more, or 1 day or more, allowing for example for a follow-up after a therapy, such as imaging sentinel lesions in sentinel lymph nodes after surgery in breast cancer.

The reconstruction protocol typically consists in reconstructing conventional images, spectral virtual monochromatic images (for example 40 keV) for increasing the attenuation of the nanoparticles (e.g. Gd chelated nanoparticles and more specifically AGuIX) and spectral K-edge when gadolinium is used as high-Z element, presenting with a K-edge at 50.2 keV for enabling a specific and quantitative tissue imaging.

In specific embodiments, said organ imaging as described herein is performed in methods for diagnosis of sentinel lymph nodes in breast cancer. In other specific embodiments, said method is performed for diagnosis and/or staging of liver and bronchopulmonary tumors. In other specific embodiments, said method is performed for the diagnosis and characterization of pathological lymph nodes.

Full Body-Scan Imaging with Injection of Two Different Atom-Based Contrast Agents

In a more specific embodiment, the method of imaging comprises the following steps of
(a) injecting intravenously an efficient amount of a pharmaceutical composition comprising the high-Z containing nanoparticles as described herein, as a first contrast agent for SPCCT, preferably gadolinium chelated nanoparticles, and more preferably AGuIX nanoparticles,
(b) injecting intravenously an efficient amount of a pharmaceutical composition comprising a second contrast agent, typically an iodinated contrast agent, for example between 10 and 20 seconds, such as for example 15 seconds, after the injection of the nanoparticles at step (a), and
(c) acquiring sequential image scans, with a first image scan acquisition at the time of injecting the second contrast agent, and one or more subsequent image scans for early and late enhancement of the organ of interest.

In specific embodiments of such method for liver imaging, scan acquisitions are performed at step b) shortly (e.g., 10-20 seconds, typically 15 seconds) after the nanoparticle injection (e.g., gadolinium chelated nanoparticles, preferably AGuIX nanoparticles) in order to acquire an arterial phase with the nanoparticle and a venous phase with the second contrast agent. A second acquisition 30 seconds after allows to acquire a portal phase with the nanoparticles and late phase with the second contrast agent. Such embodiment may be used advantageously for liver imaging

In specific embodiments for imaging of the urinary tract, scan acquisitions are performed at step b) shortly (e.g; 10-20 seconds, typically 15 seconds) after the nanoparticle injection (e.g., gadolinium chelated nanoparticles, preferably AGuIX nanoparticles) and a medullar phase with the second contrast agent. A second acquisition from 30 seconds to 90 seconds after, typically 60 seconds after allows to acquire a medullar phase with the nanoparticles and urinary phase with the second contrast agent.

In specific embodiments for peritoneal imaging, the nanoparticles (preferably gadolinium chelated nanoparticles and more preferably AGuIX nanoparticles) are injected in the peritoneal cavity, typically from 200 to 400 mg/mL, for example 300 mg/mL) and scan acquisitions are performed at step b) between 30 and 90 seconds, typically 60 seconds after the injection of the second contrast agent (e.g. iodinated contrast agent) in order to acquire a venous enhancement of the peritoneal carcinomatosis and/or the abdominal and pelvic organs. In particular, the gadolinium chelated nanoparticles, such as AGuIX nanoparticles, allows a negative contrast in the peritoneal cavity to increase the visualization of carcinomatosis (invest Radiol. 2018 October; 53(10):629-639. doi: 10.1097/RLI.0000000000000483; Sci Rep. 2020 Aug. 7; 10(1):13394. doi: 10.1038/s41598-020-70282-w). A second acquisition for example 60 seconds later is performed to evaluate the lesions of the peritoneum at the late time. This allows to benefit from the persistent negative intraperitoneal contrast brought by the injection of the nanoparticles in situ.

In specific embodiments for pleural imaging, the nanoparticles (preferably gadolinium chelated nanoparticles and more preferably AGuIX nanoparticles) are injected in the pleural cavity, typically from 200 to 400 mg/mL, for example 300 mg/mL) and scan acquisitions are performed at step b) between 30 and 90 seconds, typically 60 seconds after the injection of the second contrast agent (e.g. iodinated contrast agent) in order to acquire a venous enhancement of the pleural carcinomatosis and/or the thoracic organs. In particular, the gadolinium chelated nanoparticles, such as AGuIX nanoparticles, allows a negative contrast in the pleural cavity to increase the visualization of carcinomatosis. A second acquisition for example 60 seconds later is performed to evaluate the lesions of the pleural cavity at the late time. This allows to benefit from the persistent negative intrapleural contrast brought by the injection of the nanoparticles in situ.

In specific embodiments, said method is performed in the diagnosis of liver tumors. In other specific embodiments, said method is performed for the diagnosis and monitoring of parenchyma of liver, pancreas, kidney, spleen, heart and brain. In specific embodiments, said method is performed for the diagnosis and/or monitoring of kidney tumor. In specific embodiments, said method is performed for the diagnosis and/or monitoring of peritoneal carcinomatosis. In specific embodiments, said method is performed for the diagnosis and/or monitoring of pleural carcinomatosis.

Combining Myocardial Imaging with AGuIX and Radiotherapy of the Heart in Human

In a more specific embodiment, the method of imaging comprises
a) injecting an effective amount of the nanoparticles as described herein, preferably gadolinium containing nanoparticles, more preferably, AGuIX nanoparticles, to said subject, as contrast agent for myocardial imaging,
b) acquiring an image of the myocardium of the subject in need thereof,
c) analyzing the image, and identifying the pathological myocardium, and,
d) irradiating the pathological myocardium by radiotherapy, preferably stereotaxic radiotherapy, in the presence of an effective amount of said nanoparticles as radiosensitizing agent, e) optionally, acquiring an image of the myocardium of the subject in need thereof after step d) in view of evaluating the efficiency and tolerability of the treatment.

The skilled person will select any suitable means for acquiring an image of myocardium, taking the benefit of the nanoparticles as contrast agent. Suitable means include without limitation Magnetic Resonance Imaging (MRI) or scanner.

In specific embodiments, at step b) and optionally at step e), the image is acquired via SPCCT scanning, MRI or scanner, preferably via SPCCT scanning.

In specific embodiments, from 100 mg/mL to 400 mg/mL of nanoparticles, preferably gadolinium containing nanoparticles, more preferably, AGuIX nanoparticles, are injected for example in an intracoronary catheter, with a flow rate of e.g. 1 mL/s, a dose adapted to the patient's weight (typically 1 mL/kg). The coronary territory in which the product is injected should correspond to the territory vascularizing the pathological myocardium, after analysis of the coronary dominance. In such embodiments, the image is acquired for example 10, 15, 20, 25, or 30 minutes after the injection, typically 20 minutes, in order to highlight the pathological myocardium.

Alternatively, from 200 mg/mL to 400 mg/mL of nanoparticles, preferably gadolinium containing nanoparticles, more preferably, AGuIX nanoparticles, are injected intravenously for example, with a flow rate of between 2 to 3 mL/s, typically 2.5 mL/s with a dose adapted to the patient's weight (2.5 mL/kg). In such embodiments, the image is acquired for example 10, 15, 20, 25, or 30 minutes after the injection, typically 20 minutes, in order to highlight the myocardial lesions.

In specific embodiments, at step c), the images are analysis for segmentation of the zone for irradiation, typically the damage tissue, and calculation of the irradiation dose.

At step d), the radiotherapy session will consist in a conventional scanner, a dual energy scanner, a multi-energy scanner with SPCCT photon counting and/or MRI depending on the radiotherapy machine. For example, a radiation dose may consist of a single dose of 25 Gy (https://doi.org/10.1136/openhrt-2021-001770), which can be fractionated in multiple doses.

Such method is particularly appropriate to improve the segmentation of the myocardial lesions and target more precisely the damage tissue for radiotherapy. The imaging step also enables to optimize the irradiation dose as function of the lesion size, concentration of the nanoparticles, for example AGuIX, for a personalized treatment.

The method is useful in particular for treating of scar of myocardial infarction, arrhythmogenic fibrosis of cardiopathy, or cardiac tumors (primary or metastasis).

The method is also useful for the preventive treatment for reperfusion lesion of myocardial infarction at the acute phase.

### EXAMPLES

### Example 1: In Vitro SPCCT Imaging with AGuIX Nanoparticles

### In Vitro SPCCT Imaging

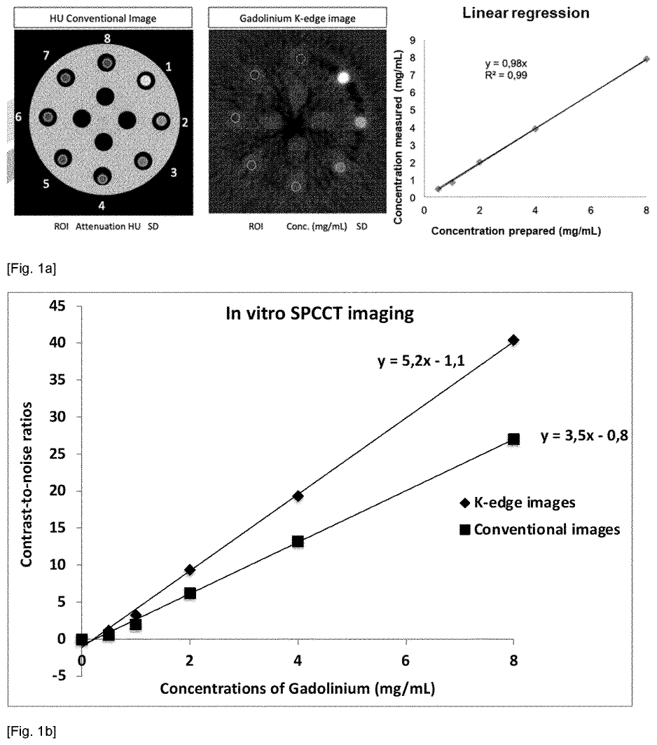

Conventional and K-edge images of 5 tubes containing AGuIX nanoparticles in Hounsfield units have been acquired at increasing concentrations of gadolinium atoms, respectively with 0.5-1-2-4-8 mg/mL. As shown in FIGS. 1a and 1b, and Table 1 below, K-edge image, unlike conventional image, collects only the signal from the gadolinium-filled tubes. The water tubes (number 6 to 8) as well as the material of the circular phantom are therefore not collected in K-edge imaging. Quantification in K-edge imaging shows a very high linear correlation.

TABLE 1
CNR ratio increases with K-edge in comparison with conventional
images as a function of gadolinium concentrations in the tubes.
K-edge imaging allows to drastically increase the CNR of the
tubes thanks to the specificity of this technique.

| Tubes No | [C.]° Gd | Increase in CNR with K-edge(%) |
|---|---|---|
| 1 | 0 | 0.00 |
| 2 | 0.5 | 122.19 |
| 3 | 1 | 66.02 |
| 4 | 2 | 50.10 |
| 5 | 4 | 46.10 |
| 6 | 8 | 49.67 |

The benefits showed by combining AGuIX and conventional images are linear attenuation of AGuIX tubes in Hounsfield units. The limitations of this association are:
No specificity,
Insufficient contrast with soft tissue for low concentrations,
No absolute quantification of gadolinium atoms (heavy metals).

Benefits showed by combining AGuIX and K-edge images are:
Signal specificity for gadolinium atoms in K-edge images: neither the phantom nor the water tubes were visible in K-edge images (FIG. 1a)
Improvement in contrast: average relative increase of 50% with K-edge images compared to conventional images (Table)
Accurate and linear quantification: curve at 0.98 and R² at 0.99 (Graph)
   NB: Contrast-to-noise was calculated as follows: ([value in analyzed tube]−[value in water tube]/value of noise in water tube].
   Iso-Osmolar Suspension of AGuiX with 43 mg/mL of Gd Atoms

The AGuIX composition exhibits an Osmolarity at 382 mosm at 21° C., i.e., iso-osmolar to blood osmolarity (300 mOsm/g H₂0). For comparison, Dotarem® (gadoteric acid, GUERBET, FRANCE) is hyperosmolar at 1574 mosm, which can cause side effects in humans related to the hypertonicity of a hyperosmolar product creating an intense call of water from the interstitial and cellular spaces to the plasma, responsible for pain or heat. A pain threshold of 600 mOsm/kg has been set.

FIG. 2 shows a first pass aortic phase SPCCT angiography in conventional image showing a high image quality.

### Example 2: Arterial and Venous SPCCT Angiography (Conventional, Spectral Virtual Monochromatic And K-Edge Images)

Benefits of combining AGuIX and SPCCT imaging are:
Performing a first-pass arterial phase SPCCT angiography with high image quality
Performing a second-pass arterial phase SPCCT angiography with high image quality up to 3 minutes.

FIG. 2 shows a Longitudinal follow up of aortic opacification in conventional image showing a strong enhancement in the aorta (white head arrows) during the first pass phase, i.e., 8 seconds after injection, until 180 seconds. In comparison to the pre-injection phase where the aorta was not observable (empty white head arrow), the aorta can still be observable until 40 minutes (full white head arrows).

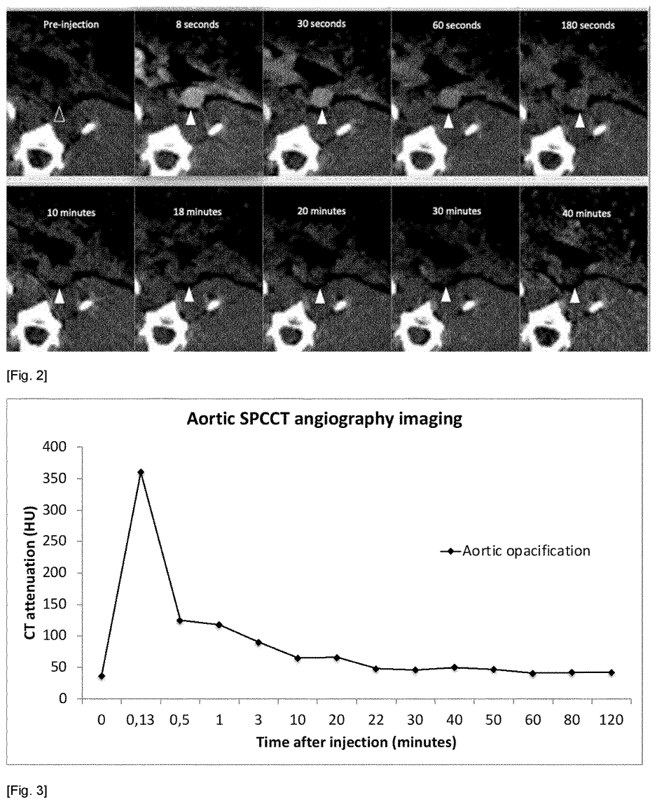

FIG. 3 shows a quantitative analysis of CT attenuation within the aorta in conventional image showing an attenuation up to about 350 HU during the first pass phase 8 seconds after injection then a persistent second pass phase up to 3 minutes with an attenuation around about 100 HU.

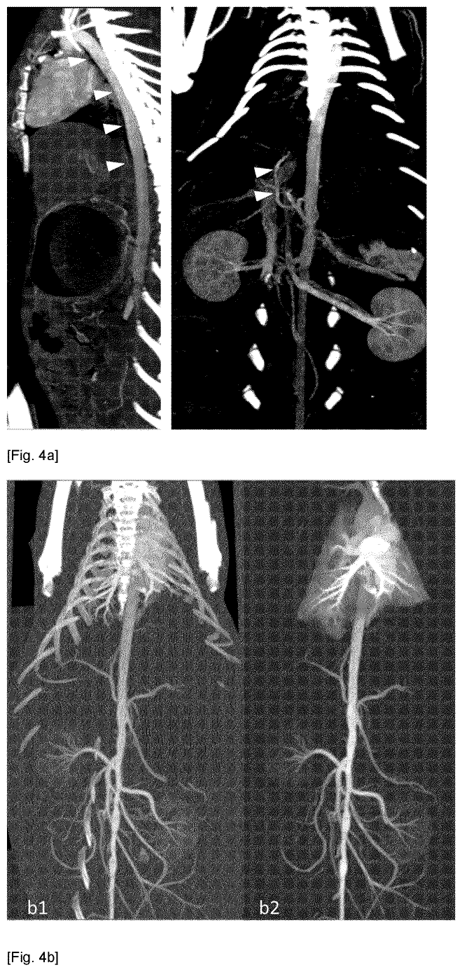

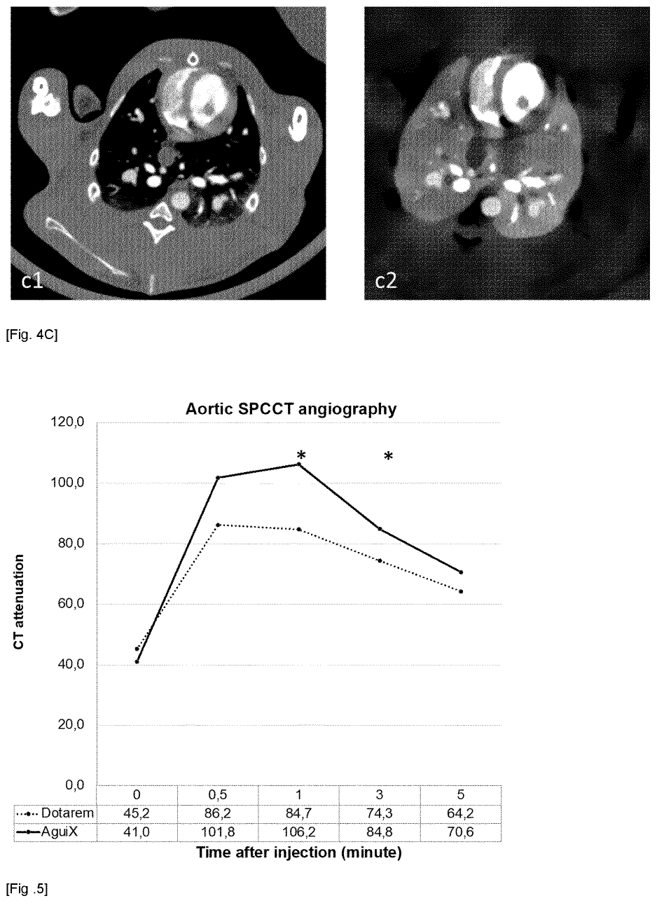

FIG. 4a shows first pass aortic phase SPCCT angiography in conventional image, showing a high image quality. FIG. 4b shows another first pass aortic phase SPCCT angiography in conventional image, showing a high image quality, and K-edge image. FIG. 4c shows cross sections of FIG. 4b. Contrary to conventional images, K-edge reveals the specific imaging of the arterial vessels, while improving the contrast of all enhanced structures (lung, kidney).

### Example 3: Comparative Study Between AGuIX and Gadolinium Chelates (Gadoteric Acid: Dotarem, Guerbet, France) for Aortic SPCCT Angiography in an Atherosclerotic Rabbit Model

Paired comparisons were performed of aortic attenuations in aortic SPCCT angiographies in rabbits injected successively with Dotarem® (gadoteric acid, GUERBET, France) and AGuIX 2 weeks apart before and after injection at 30 seconds, 1, 3 and 5 minutes. The results are shown in FIG. 5.

For the same gadolinium atom loadings, the results showed similar high quality aortic attenuation immediately after injection and then significantly higher image quality with AGuIX at successive times. Aortic opacification was significantly higher at the following times: 30 seconds, 1, and 3 minutes after injection allowing for an extended time imaging window. Morphological imaging of the atherosclerotic aorta was better with AGuIX. The results showed also a higher opacification within the venous system in a prolonged manner due to the higher blood circulation.

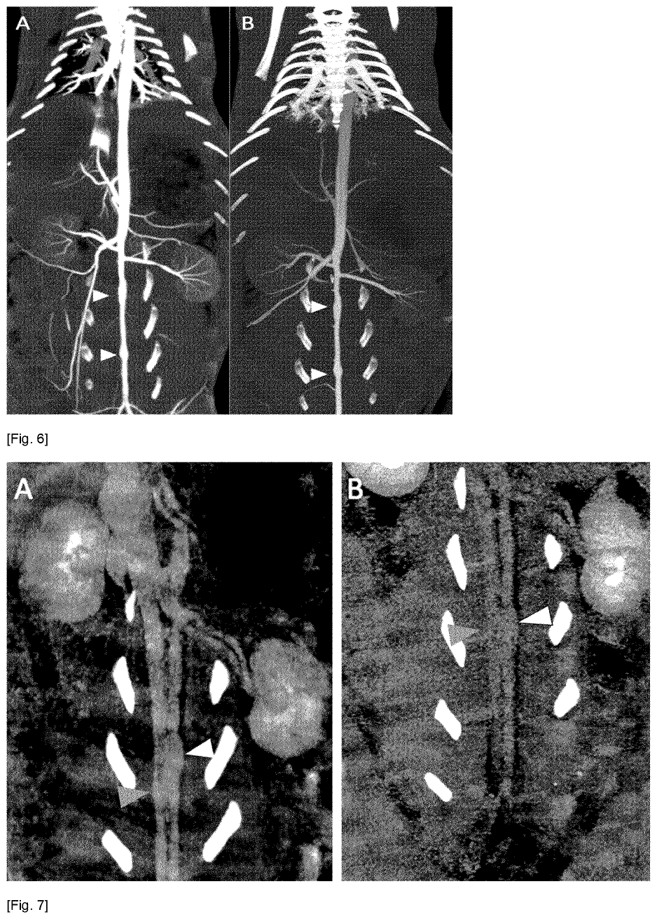

FIG. 6 shows aortic SPCCT angiography was performed during first pass arterial phase using AGuIX (A) versus Dotarem® (gadoteric acid, GUERBET, France) in an atherosclerotic rabbit model. Conventional images using AGuIX demonstrated a higher image quality than images with Dotarem®, with a clearer visualization of the aneurysmal aortic part.

FIG. 7 shows Aortic SPCCT angiography during second pass arterial phase using AGuIX (A) versus Dotarem® (gadoteric acid, GUERBET, France) in an atherosclerotic rabbit model. Conventional images using AGuIX demonstrated a higher image quality than images with Dotarem®, with a clearer visualization of the aneurysmal aortic part (red arrows) as well as for the inferior vena cava visualization (blue arrows).

### Example 4: Coronary Artery Imaging in Pigs

Coronary SPCCT angiography with heart rate synchronization was performed in 7 pigs with an average weight of 35 kg for coronary artery. Intra-arterial injection of 2 mL/s and 10 ml volume of AGuIX concentrated at 15 mg/mL Gd atoms was performed within the left coronary common trunk.

Opacification within the left descending artery was measured above the minimum threshold on conventional images (i.e, ~300 HU) required for satisfactory diagnostic quality, while it was increased up to 660 HU on 40 keV virtual monochromatic images. Only K-edge imaging allowed quantification of gadolinium concentration within the coronary arteries (~4-6 mg/mL).

The results at the arterial phase highlight the feasibility of coronary imaging in SPCCT allowing to benefit from the spectral low virtual monochromatic images in order to increase the opacification of the coronary arteries and thus either decrease the total volume of contrast medium or significantly enhance the signal.

These results particularly show the feasibility of K-edge imaging in combination with AGuIX to provide specificity in the visualization of the coronary lumen which would open the way to a better quantification of the arterial lumen in the presence of stent or calcification.

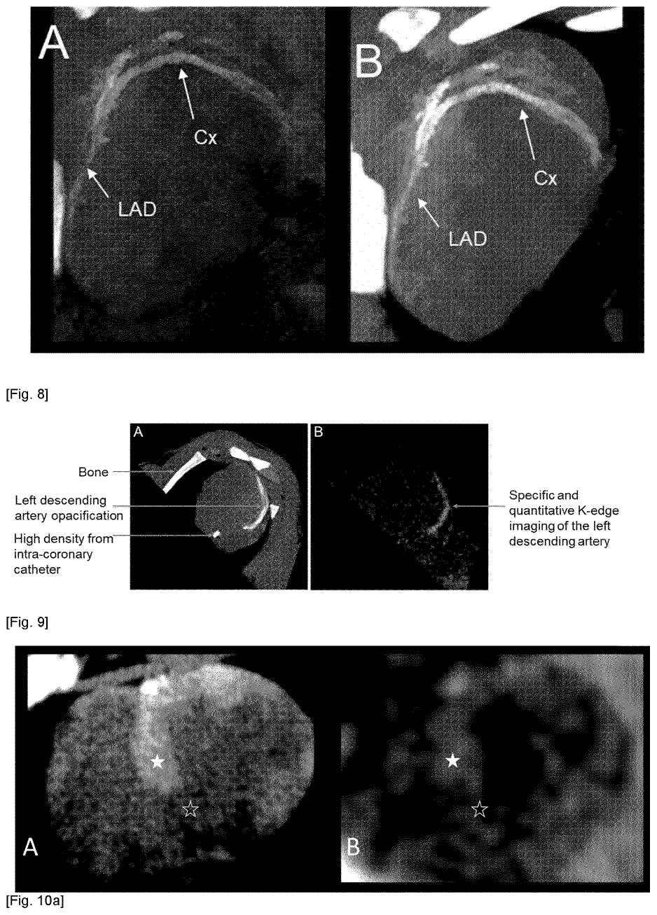

FIG. 8 shows coronary SPCCT angiography after intra-arterial injection of 10 ml AGuIX injected at 2 mL/s concentrated at 15 mg/mL of Gd atoms. Spectral material decomposition image into water and iodine (A) and spectral virtual 40 keV monochromatic image show strong opacification of the left descending artery (LAD) and the circumflex artery (Cx).

FIG. 9 shows coronary SPCCT angiography after intra-arterial injection of 10 mL AGuIX injected at 2 mL/s concentrated at 15 mg/mL of Gd atoms. Conventional images (A) show opacification of the left descending artery as well as other tissue such as bone, muscle and intra-arterial catheter. While K-edge images (B) show specifically the distribution of AGuIX within the left descending artery.

### Example 5: Arterial Myocardial Muscle Imaging in Dias

Myocardial muscle SPCCT imaging during first pass arterial phase during a coronary SPCCT angiography with heart rate synchronization was performed in 7 pigs with an average weight of 35 kg for coronary and myocardial evaluation of ischemia and infarction.

The results show a first-pass enhancement within the healthy muscle under the dependence of the opacified artery (i.e., left descending artery), whereas within the muscle under the dependence of the right coronary (that is not opacified), there is no enhancement noticeable (FIG. 10).

FIG. 10a shows first pass arterial myocardial muscle SPCCT imaging after injection of 10 mL AGuIX injected at 2 mL/s concentrated at 15 mg/mL of Gd atoms into the coronary let trunk with conventional (A) and K-edge (B) images. On conventional image, enhancement of the muscle vascularized (full white star) as well as the myocardial muscle that is not vascularized (white empty star), bone and sub cutaneous muscle are seen while on K-edge image, only the enhancement in the vascularized myocardial (full white star) muscle is seen. Concentrations of gadolinium were measured of 4 mg/mL in the vascularized myocardium and of 0 in the non-vascularized one.

This demonstrates the feasibility of myocardial muscle imaging, in particular during arterial phase, opening to a quantitative and specific perfusion analysis with the use of K-edge imaging.

FIG. 10b has been obtained in the same way. The highest definition of the images were gained by a similar to the procedure of example 11 below (invasive coronary angiography).

### Example 6: Late Myocardial Muscle Imaging in Dias and Evaluation of Infarction

Late myocardial muscle imaging at 10 and 20 minutes after intra-arterial injection of AGuiX, via cardiac CT scan as done in humans, was performed in 7 pigs with an average weight of 35 kg for myocardial evaluation of infarction.

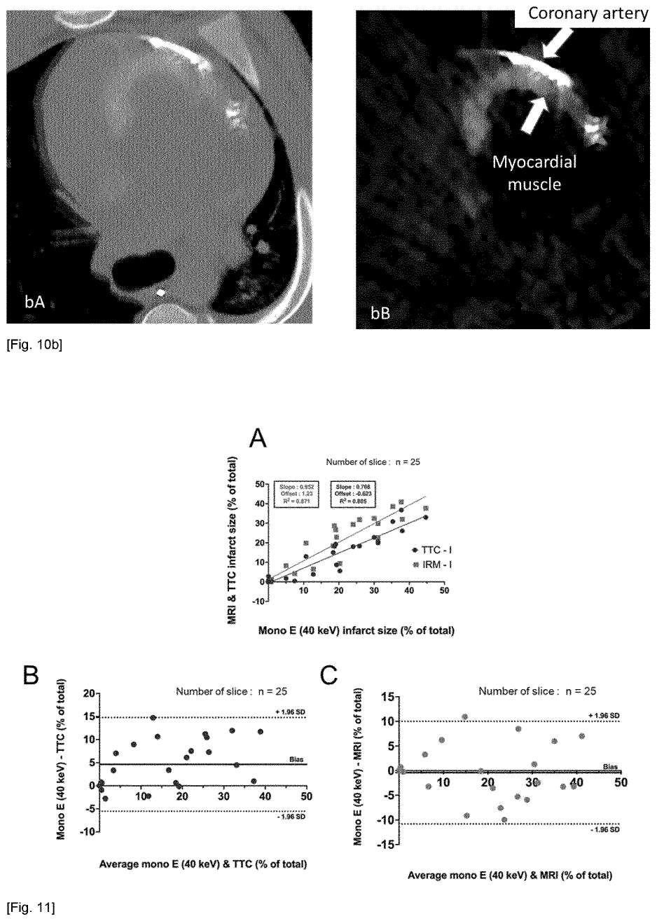

The results show a late enhancement within the infarcted muscle whereas within the healthy area the enhancement is significantly decreased (FIG. 11). Analysis of K-edge images allowed quantification of gadolinium concentration within the infarct opening to analysis of quantitative biomarkers such as for extracellular volume. This biomarker allows a quantitative analysis of the infarct opening to its better and accurate evaluation, to a better inter- and intra-patient comparability and will become a clinical standard for the evaluation of infarction and other heart diseases.

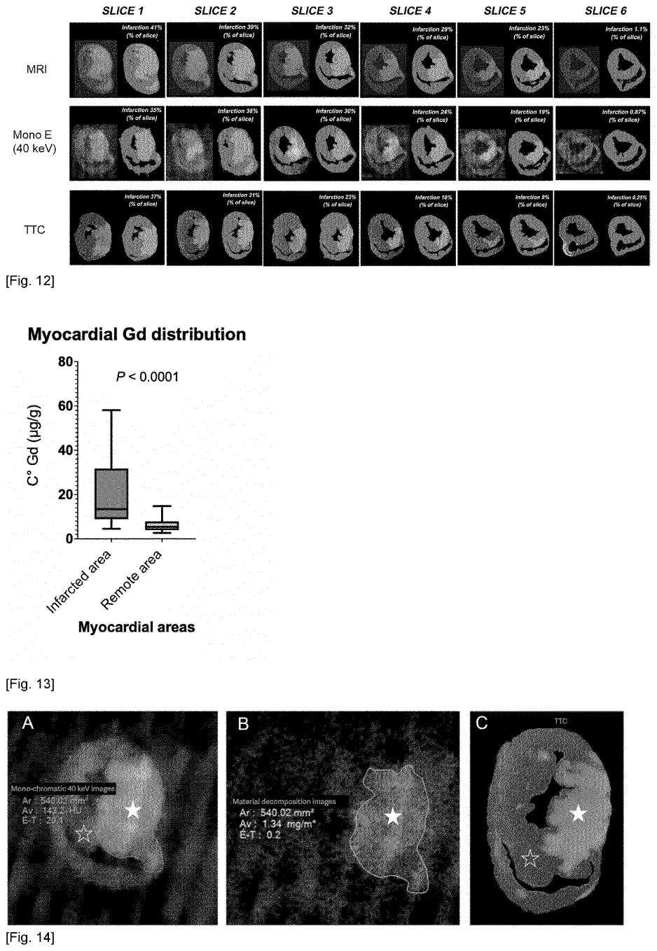

In this study, we performed ex vivo MRI and staining of the infarcted area as method of reference to compare with SPCCT imaging. The analysis found accurate agreement between 40 keV monochromatic images and MRI (R2=0.87) as well as TTC labeling (R2=0.88), validating SPCCT imaging (Figure). The concordance analysis showed an overestimation of the infarct size on SPCCT images in comparison to staining (Figure). This is explained by the difference of the 2 methods for identifying the infarction and is well known for MRI with gadolinium chelates. Mile the concordance analysis showed similar performances between SPCCT and MRI (FIG. 12). This similarity is explained by the paramagnetic characteristics of the AGuIX while it can produce also attenuation for SPCCT. Hence, the signal is co-localized between MRI and SPCCT for the same lesion.

As can be seen on FIG. 11, linear regression (A) analysis showed an excellent correlation of the infarct size between the spectral 40 keV virtual monochromatic images and MRI as well as with staining with triphenyltetrazolium chloride (TTC). Bland Altman graphs (B, C) showed negligible bias limits of agreement between spectral 40 keV virtual monochromatic images and histology as well as with staining with triphenyltetrazolium chloride (TTC).

As can be seen on FIG. 12, head-to-head comparison of MRI (first row), SPCCT (second row) and triphenyltetrazolium chloride (TTC) images showed a high qualitative and quantitative reproducibility for myocardial infarction imaging on an ex vivo pig model.

Quantification of Gd atoms on ex vivo samples within infarcted and healthy muscle showed significantly higher values within the infarcted areas in comparison as remote areas as expected, confirming the imaging findings (FIGS. 13 and 14).

This result is explained by an accumulation of AGuIX within the infarcted zone, particularly within the extracellular space of the necrotic myocardial tissue. This accumulation persists over time due to the pathophysiology of the infarction in the acute phase, i.e., due to edema that decreases the capacity of elimination of contrast products in a transient manner.

### Example 7: Kidney and Urinary Tract Imaging in Rabbits

Kidney and urinary tract follow-up imaging was performed on 9 rabbits before and after IV injection of AGuIX (concentrated at 43 mg/mL of Gd atoms) at 2.5 mL/s at 8 seconds, 30 seconds, 1, 3 and 5 minutes. Results were compared to an injection of Dotarem® (gadoteric acid, GUERBET, France) so the Gd atoms load was the same than with AGuIX injection.

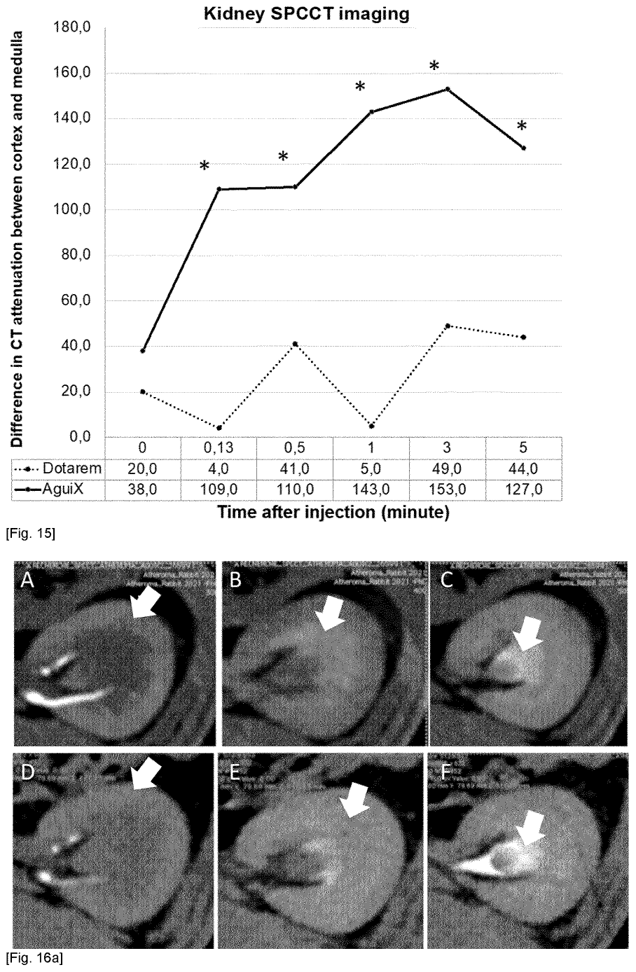

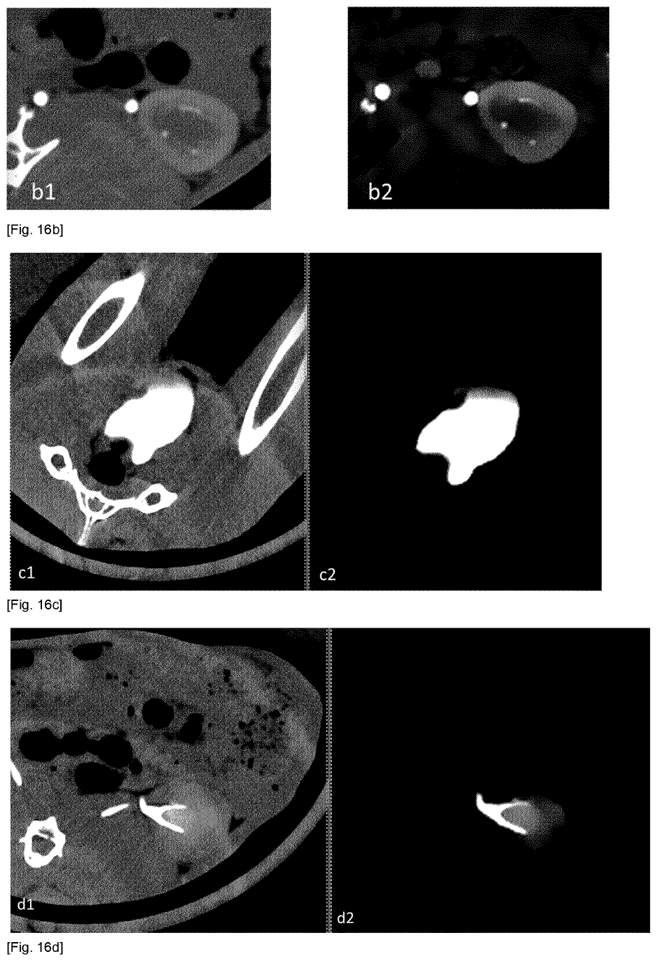

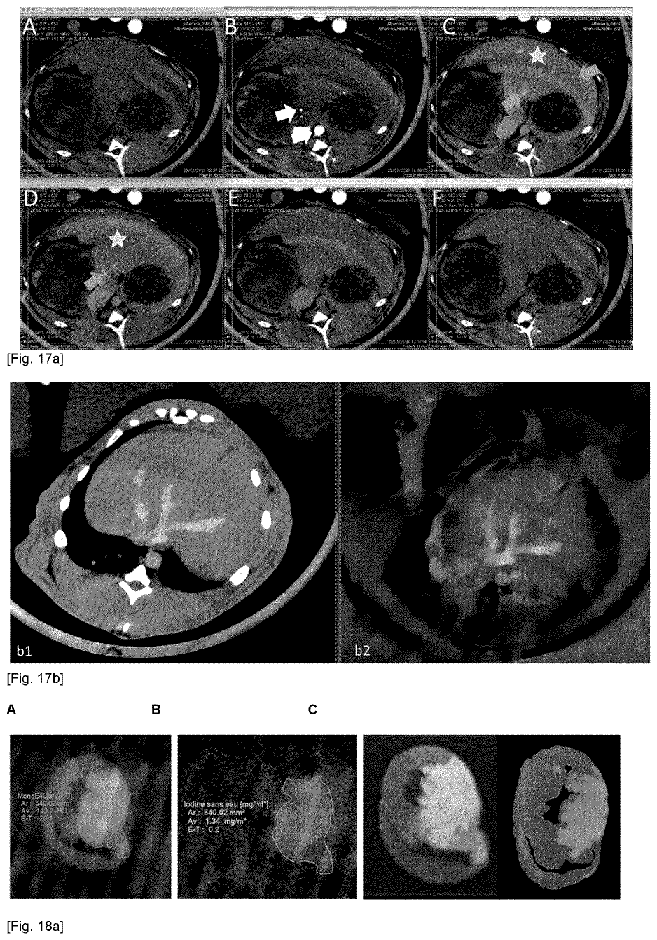

The results showed a higher contrast in attenuation between the cortex and the medulla of the kidney opening to a better functional assessment of the kidney and better visualization of renal failure (FIG. 15). In addition, the contrast in kidney was maintained at least during 5 minutes which allows to increase the time imaging window (FIG. 16a).

As can be seen on FIG. 15, comparison of the difference in attenuation (Delta attenuation) between cortex and medulla as function of time and contrast medium used: in black dotted line, Dotarem® (gadoteric acid, GUERBET, France); in black line, AGuIX. The large delta with AGuIX allows for an improved differentiation of the cortex from the medulla for optimized renal and urinary tract imaging, in comparison to the other contrast agent.

As can be seen on FIG. 16a, kidney and urinary tract SPCCT imaging with AGuIX (top row; A-C) compared with Dotarem® (gadoteric acid, GUERBET, France) (bottom row; D-F). Conventional images show high quality image for urinary tract and kidney imaging and demonstrate a better differentiation of the cortex (A and D, white arrows) at 8 seconds after injection, of the medulla (B and E, white arrows) at 30 seconds and of the pelvic urinary tract at 5 minutes (C and F, white arrows).

FIGS. 16b (kidney), 16c (bladder) and 16d (urinary tract) are conventional images (b1, c1, d1) and K-edge images (b2, c2, d2). Contrary to conventional images, Kedge reveals the specific imaging of the kidney perfusion, the specific imaging of the bladder (20 minutes after injection) and specific imaging of urinary tract at a urinary tract phase (20 minutes after injection) while improving the contrast of all enhanced structures.

### Example 8: Liver Imaging in Rabbits

Liver follow-up imaging was performed on 9 rabbits before and after IV injection of AGuIX (concentrated at 43 mg/mL of Gd atoms) at 2.5 mL/s at 8 seconds, 30 seconds, 1, 3 and 5 minutes.

The results showed a high attenuation in the arterial vascularization of the liver, then a homogeneous enhancement of the portal vein and the liver parenchyma up to 1 minute. This may enable the imaging of a liver cancer.

As can be seen on FIG. 17a, liver follow-up imaging performed after IV injection of AGuIX (43 mg/mL Gd atoms, 2.5 mL/s), showing successive imaging of arteries (white arrows), veins (grey arrows), and liver parenchyma (white star) up to 1 minute.

FIG. 17b shows another liver SPCCT angiography in conventional image (b1), showing a high image quality, and K-edge image (b2). Contrary to conventional images, K-edge reveals the specific imaging of the liver enhancement at 60 seconds after injection.

### Example 9: Bimodal SPCCT and MRI Imaging

Because of the paramagnetic characteristic of AGuIX and the linear absorption coefficient of the gadolinium atoms, it is possible to use AGuIX as a bimodal contrast agent for MRI and SPCCT.

### Bimodal Myocardial Infarction Imaging

FIG. 18a shows myocardial infarction in an ex vivo pig heart with 40 keV SPCCT virtual monochromatic image (A), spectral SPCCT material decomposition image (B), and T1-weighted sequence MRI imaging (C), in comparison with histological reference triphenyltetrazolium chloride (TTC) staining. Imaging on SPCCT images are concordant with MRI and TTC, claiming the feasibility to identify myocardial infarction.

FIG. 18b shows another myocardial infarction in an ex vivo pig heart SPCCT conventional image (bA), K-edge image (bB), and superposition of bA and bB (bC). Contrary to conventional images, Kedge reveals the specific imaging of myocardial infarction.

MRI provided similar results to conventional staining and SPCCT for the visualization and quantification of the infarction size.

These findings demonstrate the feasibility of myocardial muscle and infarction imaging together with cardiac MRI.

### Bimodal Aortic Imaging

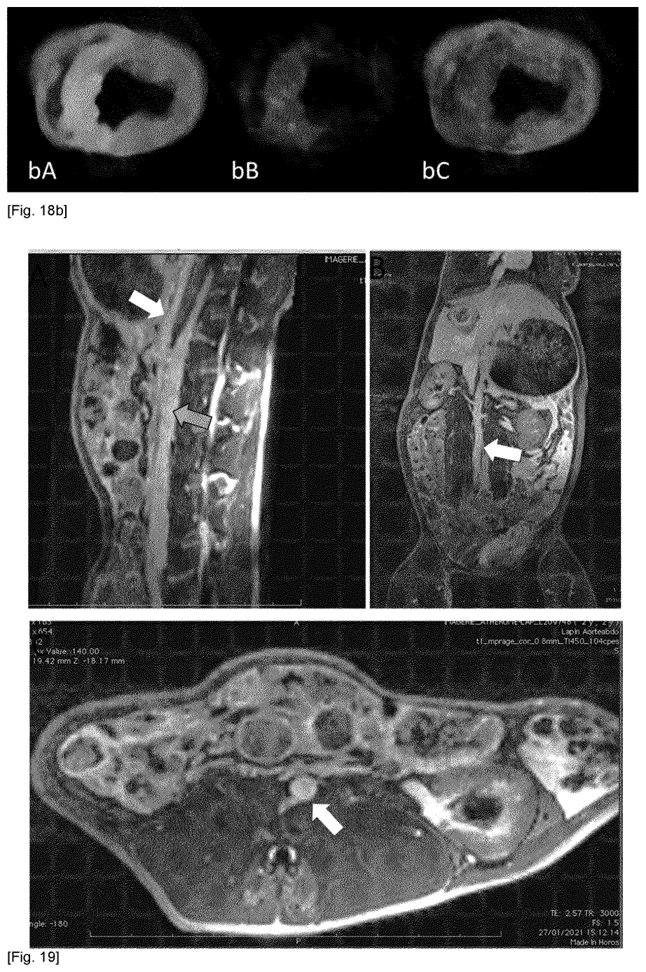

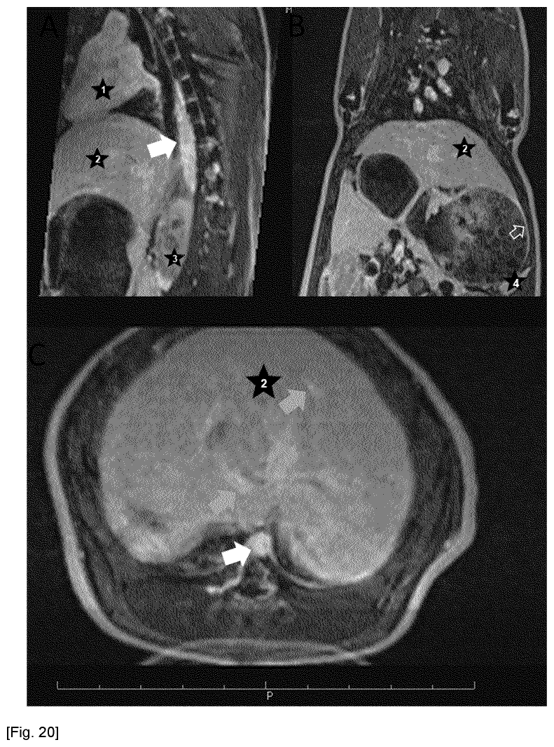

FIG. 19 shows 3D T1-weighted MRI imaging 3 hours after injection of AGuIX for aortic imaging (white arrows) after injection of 43 mg/mL Gd atoms at 2.5 mL/s. Satisfactory enhancement of the vena cava (grey arrow) is also noted.

MRI provided similar results to SPCCT for the visualization of the aortic lumen with a possibility to provide high signal within the vessels during at least 3 hours after injection (FIG. 19).

### Bimodal Liver Imaging

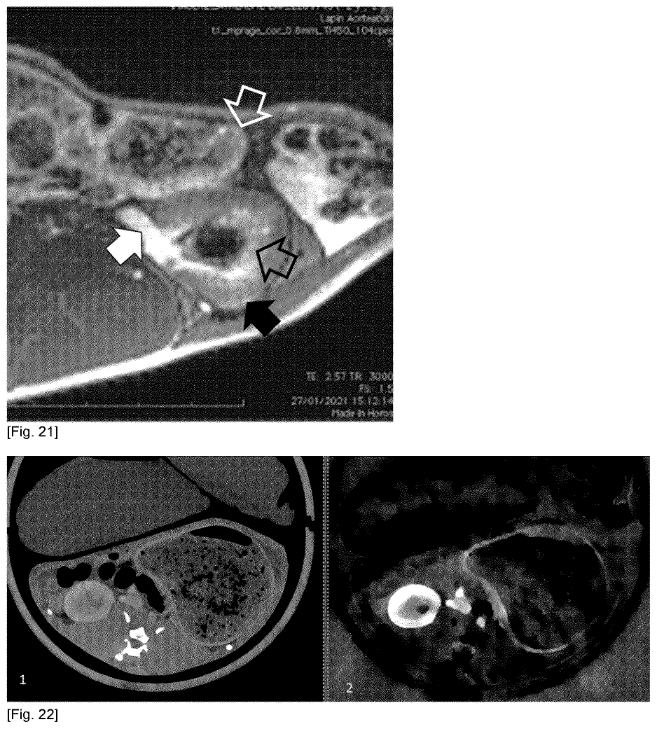

FIG. 20 shows 3D liver T1-weighted MRI imaging 3 hours after injection of AGuIX (concentrated at 43 mg/mL Gd atoms, injected at 2.5 mL/s) showing good visualization of the liver parenchyma (star 2), the portal and hepatic veins (grey arrows). Satisfactory enhancement of digestive walls (white empty arrow), kidney (star 3), spleen (star 4), heart (star 1) and vessels (white arrow) is also noted.

Liver MRI was performed 3 hours after injection, showing a persistent signal in the liver blood vessels and liver parenchyma. The image quality was sufficient for diagnostic use of potential liver tumor or disease.

### Bimodal Kidney and Urinary Act Imaging

FIG. 21 shows 3D kidney T1-weighted MRI imaging 3 hours after injection of AGuIX (concentrated at 43 mg/mL Gd atoms, injected at 2.5 mL/s) showing good visualization of the cortex (blue arrow); the medulla (purple arrow) and urinary tract (white arrow). Satisfactory enhancement of digestive walls (green arrow) is also noted.

Kidney and urinary tract was performed 3 hours after injection in MRI, showing a persistent signal in the urinary tract, as in the medulla and cortex. The image quality was sufficient for diagnostic use of potential tumor or disease.

### Example 10: Full Body-Scan Imaging after Intravenous Injection of AGuIX in Human

### Injection and Acquisition Protocol

Injection of 300 mg/mL AGuIX is performed intravenously in a human subject, via a peripheral and/or central catheter, at a flow rate of 2.5 mL/s, of a volume adjusted to the patient weight (between 1.5 to 2.5 mL/kg).

Scan acquisitions are performed 15 seconds after the end of injection for an arterial phase imaging, 60 seconds after injection for an arteriovenous imaging and for the enhancement of the thoracic, abdominal and pelvic organs. A late phase scan acquisition is performed 3 minutes after injection for evaluating the wash out in the organs and also for taking benefit of the delayed time imaging window into the blood compartment.

For oncologic imaging, late scan acquisitions at 40 minutes, 1 hour, 2 hours, 3 hours and 1 one day are performed for following the enhancement kinetics and for performing a post-operative or post-radiotherapy imaging without the need to re-inject due to the extended time imaging window.

### Reconstruction Protocol

Consisting in reconstructing conventional images, spectral virtual monochromatic images (for example 40 keV) for increasing the attenuation of AGuIX and spectral K-edge of the gadolinium 50.2 keV for enabling a specific and quantitative tissue imaging.

### Clinical Indications

Diagnosis and follow-up of arterial and venous diseases
Diagnosis and follow-up of oncologic diseases
Diagnosis and follow-up of inflammatory disease
Diagnosis and follow-up of traumatic disease

### Example 11: Cardiac Imaging after Intra-Arterial Injection of AGuIX in Human in the Context of Coronary Artery Disease or Acute Chest Dain or Acute Coronary Syndrome

The method for cardiac imaging in a human subject essentially consists of
Injecting AGuIX into the aortic root and/or coronary arteries in a human subject suffering from coronary artery disease or acute chest pain or acute coronary syndrome, and,
Performing heart imaging during the late phase of myocardial enhancement from few minutes after injection to 7 days

### Injection and Acquisition Protocol

Injection of 100 to 300 mg/mL AGuIX is performed in the coronary arteries or in the aortic root during an invasive coronary angiography, via a catheter, at a flow rate between 5 and 15 mL/s, of a volume independent from the patient weight. Volume is adjusted to the distribution volume inside the coronary arteries or the aortic root. Injection of AGuIX enables a coronary angiogram in the cathlab. Consequent to the AGuIX distribution within the myocardial muscle, this will enable a short-imaging of the myocardial muscle in the SPCCT room for evaluating the myocardial damages. Additionally, the patient will undergo a cardiac MRI at mid-term for a complementary baseline evaluation of the infarct size.

### Reconstruction Protocol

Consisting in reconstructing conventional images, spectral virtual monochromatic images (for example 40 keV) for increasing the attenuation of AGuIX and spectral K-edge of the gadolinium, presenting a K-edge at 50.2 keV for enabling a specific and quantitative tissue imaging.

### Clinical Indications

Diagnosis and follow-up imaging of the size and involvement of reperfusion lesion occurring during the acute phase of a myocardial infarction
Diagnosis and follow-up imaging of the size of myocarditis
Diagnosis and follow-up imaging of aortic lesions that can be suspected after an invasive coronary angiography (ulcer, aortic dissection, intramural hematoma). This can occur when the clinical presentation is mimicking an acute coronary syndrome.

### Example 12: Brain Imaging after Intra-Arterial Injection of AGuIX in Human

The method essentially consists of
Injecting AGuIX into cerebral arteries during an endovascular mechanical thrombectomy, and,
Performing a brain imaging in order to benefit from the AGuIX injection.

### Injection and Acquisition Protocol

Injection of 100 to 300 mg/mL AGuIX is performed in the cerebral arteries during an invasive angiography, via a catheter, at a flow rate between 5 and 15 mL/s, of a volume independent from the patient weight. Volume is adjusted to the distribution volume inside the coronary arteries or the aortic root. Injection of AGuIX enables a cerebral angiogram in the cathlab. Consequent to the AGuIX distribution within the brain and the permeability of the blood brain barrier during an acute stroke, this will enable a short-imaging of the stroke in the SPCCT room for evaluating the brain damages, and also for differentiating the necrosis from the hemorrhage (Brain Commun. 2020 Nov. 11; 2(2):fcaa193.doi: 10.1093/braincomms/fcaa193). Additionally, the patient will undergo a brain MRI at mid-term for a baseline evaluation of the infarct size.

### Reconstruction Protocol

Consisting in reconstructing conventional images, spectral virtual monochromatic images (for example 40 keV) for increasing the attenuation of AGuIX and spectral K-edge of the gadolinium 50.2 keV for enabling a specific and quantitative tissue imaging.

K-edge imaging will be particularly targeted for imaging the blood brain barrier permeability after stroke and differentiating from hemorrhage after mechanical thrombectomy.

### Clinical Indications

Diagnosis and follow-up of stroke size and involvement of hemorrhage within the necrosis
Diagnosis and follow-up of blood brain barrier permeability after mechanical thrombectomy for stroke or for inflammatory, infectious and tumoral lesions.

### Example 13: Cardiovascular Imaging with Extended Imaging Time Window in Human

The method essentially consists in
injecting AGuIX through the artery or the vein in a human subject in need thereof, and
performing serial acquisition at different time points for cardiovascular system imaging
   Injection and Acquisition Protocol after IV Injection

Injection of 300 mg/mL AGuIX is performed intravenously, via a peripheral and/or central catheter, at a flow rate of 2.5 mL/s, of a volume adjusted to the patient weight (between 1.5 to 2.5 mL/kg).

Scan acquisitions are performed from 15 seconds to 3 minutes after the end of injection for an imaging of the coronary arteries, neck and cerebral arteries, aorta and its branches. Coronary arteries will be acquired during a ECG-gated acquisition.

Injection and Acquisition Protocol after Intra-Arterial Injection

Injection of 100 to 300 mg/mL AGuIX is performed in the coronary arteries or the aortic root in the SPCCT room, via a catheter localized in an artery of interest, at a flow rate between 2 and 15 mL/s, of a volume independent from the patient weight. Volume is adjusted to the distribution volume of the artery of interest.

Scan acquisitions are performed 15 seconds after the end of injection and can be performed up to 3 to 5 minutes for taking benefit of the extended time imaging window of the blood compartment.

### Reconstruction Protocol

Consisting in reconstructing conventional images, spectral virtual monochromatic images (for example 40 keV) for increasing the attenuation of AGuIX and spectral K-edge of the gadolinium 50.2 keV for enabling a specific and quantitative tissue imaging.

### Clinical Indications

Diagnosis and follow-up of coronary artery disease, neck and brain arteries, aortic and its branches (medullar artery, bronchial and kidney artery for example).

### Example 14: Urinary Tract and Kidney Imaging in Human

The method essentially consists in
injecting AGuIX through the artery or the vein in a human subject in need thereof,
performing serial acquisition on the urinary tract.
   Injection and Acquisition Protocol after IV Injection

Injection of 300 mg/mL AGuIX is performed intravenously, via a peripheral and/or central catheter, at a flow rate of 2.5 mL/s, of a volume adjusted to the patient weight (between 1.5 to 2.5 mL/kg).

Scan acquisitions are performed 15 seconds after the end of injection for a cortical phase imaging, 60 seconds for a medullar phase imaging and from 3 minutes to 3 hours for a urinary tract imaging.

Injection and Acquisition Protocol after In Situ Injection

Injection of 100 to 300 mg/mL AGuIX is performed in the urinary tract, via a nephrostomy or a bladder catheter, at a flow rate 1-5 mL/s, with a volume adjusted to distribution volume of the urinary tract (between 50-1000 mL).

Scan acquisitions are performed at the end of injection in the SPCCT room for a urinary tract imaging.

### Reconstruction Protocol

Consisting in reconstructing conventional images, spectral virtual monochromatic images (for example 40 keV) for increasing the attenuation of AGuIX and spectral K-edge of the gadolinium 50.2 keV for enabling a specific and quantitative tissue imaging.

K-edge imaging allows also the differentiation between the cavities filled with AGuIX and the spontaneous hyperdensities for a better depiction of kidney stones.

### Clinical Indications

Diagnosis and follow-up of kidney stone disease
Diagnosis and follow-up of urinary tract and kidney tumors
Diagnosis and follow-up of chronic renal failure by means of the quantification of the glomerular filtration rate (Radiology. 2011 August; 260(2):414-20. doi: 10.1148/radiol.11101317)

### Example 15: Oman Imaging after In Situ Injection in Human

The method essentially consists in
injecting AGuIX into an organ of interest to take advantage of AGuIX distribution locally in a human subject in need thereof,
performing serial acquisition of the organs of interest, e.g., lymph nodes, breast cancer, bronchopulmonary cancer, liver and vascular tumors.

### Injection and Acquisition Protocol

Injection of 100 to 300 mg/mL AGuIX is performed in the urinary tract, via a nephrostomy or a bladder catheter, at a flow rate 1-5 mL/s, with a volume adjusted to distribution volume of each organ (i.e., 10-20 mL for lymphatic system, 50-1000 mL for urinary tract, 5-10 mL for brain ventricular system, 1-10 mL for the lymph nodes, 1-10 mL for the breast, bronchopulmonary, liver and vascular tumors).

Scan acquisitions are performed at the end of injection in the SPCCT room in order to show a local deposition and peripheral distribution in sentinel lesions such as lymph nodes in breast cancer.

Time points for imaging can be repeated and extended from hours to day allowing for a follow-up after therapy, such as for sentinel lymph nodes after surgery in breast cancer.

### Reconstruction Protocol

Consisting in reconstructing conventional images, spectral virtual monochromatic images (for example 40 keV) for increasing the attenuation of AGuIX and spectral K-edge of the gadolinium 50.2 keV for enabling a specific and quantitative tissue imaging.

### Clinical Indications

Sentinel lymph nodes in breast cancer
Imaging of lymphatic system (thoracic duct, accessory ducts)
Staging of liver and bronchopulmonary tumors
Diagnosis and characterization of pathological lymph nodes

### Example 16: Full Body-Scan Imaging with Injection of Two Different Atom-Based Contrast Agents Including AGuIX in Human

The method consists in
injecting AGuIX and a different atom-based contrast agent through the vein, artery or in situ, in a human subject, and,
performing serial acquisition at different time of early and late enhancement of organ of interest.

This protocol is particularly adapted to the organs with sequential phases of enhancement such as for liver, kidney, pancreas, adrenal gland, spleen, myocardium, lymph nodes, urinary tract.

It is also particularly adapted to the imaging of oncologic, ischemic, inflammatory and infectious process.

The lesions targeted by this protocol are particularly the inflammatory and tumoral lymph nodes, breast, bronchopulmonary, liver and vascular tumors.

### Injection and Acquisition Protocol Aer Multiple Contrast Agent Injection for Liver Imaging

Injection of 300 mg/mL AGuIX is performed intravenously, via a peripheral and/or central catheter, at a flow rate of 2.5 mL/s, of a volume adjusted to the patient weight (between 1.5 to 2.5 mL/kg). AGuIX injection precedes from 15 seconds an intravenous injection of iodinated contrast agent (concentration between 300 and 400 mg/mL) via a catheter with a flow rate of 2.5 mL/s, and a volume adjusted to the patient weight (1 mL/kg).

Scan acquisitions are performed 15 seconds after AGuIX injection in order to acquire an arterial phase with AGuIX and a venous phase with the iodinated contrast agent. A second acquisition 30 seconds after allows to acquire a portal phase with AGuIX and late phase with the second contrast agent.

### Injection and Acquisition Protocol Ater Multiple Contrast Agent Injection for Urinary Tract Imaging

Injection of 300 mg/mL AGuIX is performed intravenously, via a peripheral and/or central catheter, at a flow rate of 2.5 mL/s, of a volume adjusted to the patient weight (between 1.5 to 2.5 mL/kg). AGuIX injection precedes from 15 seconds an intravenous injection of iodinated contrast agent (concentration between 300 and 400 mg/mL) via a catheter with a flow rate of 2.5 mL/s, and a volume adjusted to the patient weight (1-2.5 mL/kg).

Scan acquisitions are performed 15 seconds after AGuIX injection in order to acquire a cortical phase with AGuIX and a medullar phase with the iodinated contrast agent. A second acquisition 60 seconds after allows to acquire a medullar phase with AGuIX and urinary phase with the second contrast agent.

### Injection and Acquisition Protocol Ater Multiple Contrast Agent Injection for Peritoneal Imaging

Injection of 300 mg/mL AGuIX is performed into the peritoneal cavity, via a transcutaneous catheter of a volume adjusted to peritoneal cavity (between 11 et 100 mL). Injection of iodinated contrast agent (concentration between 300 and 400 mg/mL) via a catheter with a flow rate of 2.5 mL/s, and a volume adjusted to the patient weight (1-2.5 mL/kg) is performed intravenously via a catheter.

Scan acquisitions are performed 60 seconds after iodinated contrast agent injection in order to acquire a venous enhancement of the peritoneal carcinomatosis and the abdominal and pelvic organs. AGuIX injection allows a negative contrast in the peritoneal cavity to increase the visualization of carcinomatosis (Invest Radiol. 2018 October; 53(10):629-639. doi: 10.1097/RLI.0000000000000483; Sci Rep. 2020 Aug. 7; 10(1):13394. doi: 10.1038/s41598-020-70282-w). A second acquisition 60 seconds later is performed to evaluate the lesions of the peritoneum at the late time while benefiting from the persistent negative intraperitoneal contrast brought by the injection of AGuIX in situ.

Injection and Acquisition Protocol after Multiple Contrast Agent Injection for Pleural Imaging

Injection of 300 mg/mL AGuIX is performed into the pleural cavity, via a transcutaneous catheter of a volume adjusted to pleural cavity (between 10 et 100 mL). Injection of iodinated contrast agent (concentration between 300 and 400 mg/mL) via a catheter with a flow rate of 2.5 mL/s, and a volume adjusted to the patient weight (1-2.5 mL/kg) is performed intravenously via a catheter.

Scan acquisitions are performed 60 seconds after iodinated contrast agent injection in order to acquire a venous enhancement of the pleural carcinomatosis and the thoracic organs. AGuIX injection allows a negative contrast in the pleural cavity to increase the visualization of carcinomatosis. A second acquisition 60 seconds later is performed to evaluate the lesions of the pleural cavity at the late time while benefiting from the persistent negative intrapleural contrast brought by the injection of AGuIX in situ.

### Reconstruction Protocol

Consisting in reconstructing conventional images, spectral virtual monochromatic images (for example 40 keV) for increasing the attenuation of AGuIX and spectral K-edge of the gadolinium 50.2 keV for enabling a specific and quantitative tissue imaging.

### Indications

Diagnosis and follow-up of liver tumors
Diagnosis and follow-up of parenchyma of liver, pancreas, kidney, spleen, heart and brain
Diagnosis and follow-up of kidney tumor
Diagnosis and follow-up of peritoneal carcinomatosis
Diagnosis and follow-up of pleural carcinomatosis

### Example 17: Combining Myocardial Imaging with AGuIX and Stereotaxic Radiotherapy of the Heart in Human

The method essentially consists in
injecting AGuIX through the coronary arteries
Identifying and segmenting the myocardial lesion
Irradiating the pathological tissues

The protocol also includes its use for the prevention of reperfusion injury in the acute phase of infarction. The irradiated dose can be given as a single dose or by fractionation, taking into account the prolonged distribution of AGuIX within the pathological tissues.

Injection and Acquisition Protocol after Intra-Arterial Injection

The injection of 300 mg/mL AGuIX is performed in the cath lab in an intracoronary catheter with a flow rate of 1 mL/s, a dose adapted to the patient's weight (1 mL/kg) and with an AGuIX concentration between 100 and 350 mg/mL. The coronary territory in which the product is injected corresponds to the territory vascularizing the pathological myocardium, after analysis of the coronary dominance.

The acquisition is performed at 20 minutes after injection to highlight the pathological myocardium. Considering the prolonged distribution of AGuIX within the pathological tissues, it is possible to perform an acquisition of location in the day after injection.

Injection and Acquisition Protocol after Intra-Veinous Injection

The injection of AGuIX concentrated to more than 300 mg/mL is performed intravenously through an intravenous catheter with a flow rate of 2.5 mL/s, with a dose adapted to the patient's weight (2.5 mL/kg).

The acquisition is performed at 20 minutes after injection to be able to highlight the myocardial lesion. Due to the prolonged distribution of AGuIX within the pathological tissues, it is possible to perform a tracking acquisition within one day after injection.

### Protocol of Radiotherapy

The radiotherapy session will consist in a conventional scanner, a dual energy scanner, a multi-energy scanner with SPCCT photon counting and/or MRI depending on the radiotherapy machine. The second step will be the delivery of the radiation dose which will be carried out in a single dose of 25 Gy (https://doi.org/10.1136/openhrt-2021-001770), which will be opened to dose optimization and in particular the possibility of splitting the doses.

### Reconstruction Protocol

The protocol consists in reconstructing conventional images, spectral virtual monochromatic images (for example 40 keV) for increasing the attenuation of AGuIX and spectral K-edge of the gadolinium 50.2 keV for enabling a specific and quantitative tissue imaging.

Particularly appropriate to improve the segmentation of the myocardial lesions and target more precisely the damage tissue. This will help to optimize the irradiation dose as function of the lesion size, concentration of AGuIX for a personalized treatment.

### Clinical Indications

Scar of myocardial infarction
Arrhythmogenic fibrosis of all cardiopathy
Cardiac tumors (primitive or metastasis)
Preventive treatment for reperfusion lesion of myocardial infarction at the acute phase

### Example 18: Stomach Imaging in Rabbits

Stomach imaging was performed on 9 rabbits before and after IV injection of AGuIX (concentrated at 43 mg/mL of Gd atoms) at 2.5 mL/s at 8 seconds.

As can be seen on FIG. 22, SPCCT angiography in conventional image (1), showing a high image quality, and K-edge image (b). Contrary to conventional images, K-edge reveals the specific imaging of the stomach wall enhancement at 8 seconds after injection.

What is claimed is:

1-15. (canceled)

16. A method for imaging an anatomical structure in a subject in need thereof, comprising steps of:
a) providing an injectable pharmaceutical composition comprising, as a contrast agent, at least one nanoparticle having a mean hydrodynamic diameter between 1 nm and 10 nm and comprising:
a biocompatible matrix,
at least one chelating agent covalently bound to said biocompatible matrix, and
at least one element having a Z of at least 40, chelated to at least a part of the chelating agent,
b) injecting an effective amount of said pharmaceutical composition to said subject, and,
c) acquiring a scan image of the anatomical structure of said subject, by Spectral Counting Computed Tomography (SPCCT) scanning.

17. The method according to claim 16, wherein the biocompatible matrix is a polyorganosiloxane matrix.

18. The method according to claim 17, wherein the at least one nanoparticle comprises:
polyorganosiloxane with a silicon weight ratio of at least 8% of the total weight of the nanoparticle,
chelating agent covalently bound to said polyorganosiloxane, in a proportion comprised between 4 and 200 per nanoparticle, and,
at least one element having a Z of at least 40 chelated to at least a part of the chelating agent.

19. The method according to claim 18, wherein the polyorganosiloxane has a silicon weight ratio between 8% and 50%.

20. The method according to claim 18, wherein the chelating agent is bound to said polyorganosiloxane in a proportion comprised between 4 and 80 per nanoparticle.

21. The method according to claim 16, wherein said chelating agent is one or more of DOTA, DTPA, EDTA, EGTA, BAPTA, NOTA, DOTAGA, DFO, DOTAM and DTPABA.

22. The method according to claim 16, wherein the element having a Z of at least 40 is selected from in the group consisting of lanthanum, cerium, praseodymium, neodymium, promethium, samarium, europium, gadolinium, terbium, dysprosium, holmium, erbium, thulium, ytterbium, lutetium, hafnium, tantalum, tungsten, rhenium, osmium, iridium, platinum, gold and bismuth.

23. The method according to claim 22, wherein the element having a Z of at least 40 is gadolinium or bismuth.

24. The method according to claim 16, wherein the at least one nanoparticle is a gadolinium-chelated polyorganosiloxane nanoparticle of formula: 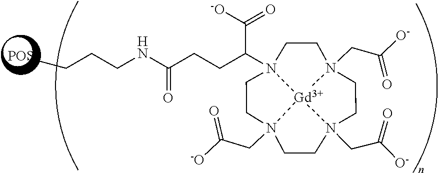
wherein POS is a matrix of polyorganosiloxane, and
n is comprised between 4 and 200, and
wherein said at least one nanoparticle has a mean hydrodynamic diameter between 2 and 8 nm.

25. The method according to claim 24, wherein n is comprised between 4 and 80.

26. The method according to claim 24, wherein said at least one nanoparticle has a mean hydrodynamic diameter between 2 and 6 nm.

27. The method according to claim 16, wherein said method is used for arterial or veinous angiographic imaging of said subject.

28. The method according to claim 27, wherein an efficient amount of said pharmaceutical composition is administered via intra-arterial or intravenous route.

29. The method according to claim 16, wherein said method is used for cardio-vascular imaging of said subject.

30. The method according to claim 16, wherein at step c) said scan image is acquired for a period of time comprised between 5 minutes and 10 minutes.

31. The method according to claim 16, wherein said method is used for diagnosing a condition in said subject, and wherein said method further comprises a step of:
d) analyzing the scan image obtained, thereby diagnosing said condition.

32. The method according to claim 31, wherein said condition is selected from the group consisting of ischemia, myocardial infarction, and stroke.

33. The method according to claim 31, wherein said condition is a myocardial infarction and said method is used for diagnosing a zone at risk and a size of an infarction before and after revascularization.

34. The method according to claim 33, wherein the pharmaceutical composition is administered in combination with an iodine contrast agent.

35. The method according to claim 16, wherein said method is used for diagnosing and treating a tumor in said subject in need thereof, and wherein said method further comprises steps of:
d) analyzing the scan image obtained, thereby diagnosing presence of said tumor, and
e) treating said tumor with said nanoparticle as a radiosensitizing agent in combination with radiation therapy.

36. The method according to claim 35, wherein said tumor is a solid tumor.

37. The method according to claim 36, wherein said solid tumor is selected from the group consisting of glioblastoma, brain metastases, meningioma, and primary tumor of uterine cervix, rectum, lung, head and neck, prostate, colorectal, liver, and pancreas.

38. The method according to claim 16, wherein said method is used for diagnosing and treating a cardiac disorder in said subject in need thereof, wherein the anatomical structure that is imaged is myocardium, and wherein said method further comprises steps of:
d) analyzing the scan image obtained, and identifying pathological myocardium, and
e) treating said subject by irradiating the pathological myocardium by radiotherapy in the presence of an effective amount of said nanoparticle as radiosensitizing agent.

39. The method according to claim 38, wherein said method further comprises a step of:
f) acquiring a scan image of the myocardium of said subject in need thereof after step e) via SPCCT scanning in order to evaluate the efficiency and tolerability of the treatment of step e).

40. The method according to claim 38, wherein in step e), irradiating the pathological myocardium is carried out by stereotaxic radiotherapy.

41. The method according to claim 38, wherein in step b) magnetic resonance imaging (MRI) is used instead of SPCCT scanning.
